(19) United States
(12) Patent Application Publication
(10) Pub. No.: US 2022/0351865 A1
(43) Pub. Date: Nov. 3, 2022

(54) METHODS OF BIOLOGICAL AGE EVALUATION AND SYSTEMS USING SUCH METHODS

(71) Applicants: Konstantin Aleksandrovich AVKHACHEV, Moscow (RU); Maksim Nikolaevich KHOLIN, Novi Sad (RS); Petr Olegovich FEDICHEV, Novi Sad (RS); Olga Andreevna BURMISTROVA, Reutov (RU); Andrei Evgenevich TARKHOV, Brookline, MA (US); Leonid Ieronimovich MENSHIKOV, Moscow (RU)

(72) Inventors: Konstantin Aleksandrovich AVKHACHEV, Moscow (RU); Maksim Nikolaevich KHOLIN, Novi Sad (RS); Petr Olegovich FEDICHEV, Novi Sad (RS); Olga Andreevna BURMISTROVA, Reutov (RU); Andrei Evgenevich TARKHOV, Brookline, MA (US); Leonid Ieronimovich MENSHIKOV, Moscow (RU)

(21) Appl. No.: 17/813,319

(22) Filed: Jul. 18, 2022

Related U.S. Application Data
(63) Continuation-in-part of application No. PCT/RU2021/050008, filed on Jan. 15, 2021.

(30) Foreign Application Priority Data
Jan. 16, 2020 (RU) 2020101622

(51) Int. Cl. G16H 50/30; G06F 17/15

(52) U.S. Cl. CPC G16H 50/30; G06F 17/15

(57) ABSTRACT

The present invention relates to methods for the determination of the biological age of a mammal and corresponding systems.

### CROSS-REFERENCE TO RELATED APPLICATIONS

This application is a continuation-in-part (CIP) of PCT Application No. PCT/RU2021/050008 filed on Jan. 15, 2021, which claims priority to Russian Patent Application No. RU2020101622 filed on Jan. 16, 2022, the contents of which are incorporated herein by reference in their entireties.

### FIELD OF TECHNOLOGY

The present disclosure relates generally to the field of biological age evaluation.

### BRIEF DESCRIPTION OF THE DRAWINGS

FIG. 1 shows a schematic of a general-purpose computer system 100 suitable for practicing the methods described herein.

FIG. 2 is a block diagram that illustrates an exemplary computer system in accordance with one or more embodiments of the present invention.

FIG. 3 is a graph illustration auto-correlation properties of age-adjusted rst PC score (z₀) in the test longitudinal dataset with sampling intervals Δt of 14 weeks (circles) and 28 weeks (squares).

FIGS. 4A and 4B illustrate principal component analysis (PCA) of the MPD slice corresponding to mature animals (age greater than 25 weeks). FIG. 4A is a graph representing the average of the PC scores in subsequent age groups where the error bars are at the standard deviation. The inset shows that the variance for all PC scores increase with age. FIG. 4B illustrates clustering of CBC features and PC scores in the training dataset. The colors represent the Pearson's correlation coefficient (absolute value) as indicated by the scale on the right side of the figure.

FIG. 5 is a table illustrating Spearman's rank-order correlation values and the corresponding p-values (in parentheses) for the top PC scores with the remaining) lifespan (the significant correlations (p<0.05) are highlighted in bold. The * marks statistically significant correlations, p<0.001.

FIG. 6 illustrates auto-correlation properties of age-adjusted dFI across sampling intervals Δt of 14 weeks (circles) and 28 weeks (squares). The * marks statistically significant correlations, p<0.001.

FIG. 7 illustrates dynamical frailty index (dFI) as a function of age in the test experiments: MA0071 (males, orange diamonds), MA0071 (females, circles) and MA0072 (triangles). The curved dashed line is the exponential fit in the age groups younger than the average lifespan of NIH Swiss mice (indicated by the dashed vertical line). The stars mark the average dFI in age-matched groups of frail animals from the MA0073 cohort. All data are presented as mean±SEM.

FIG. 8A is a graph illustrating correlation of dFI with the physiological frailty index (PFI) and FIG. 8B is a graph illustrating the extended set of phenotype measures in the test datasets MA0071 and MA0072. Features with correlation above and below significance level 0.001 are shown with grey and blue circles, respectively. The most significant correlations (excluded dFI components) were between dFI and -reactive protein (CRP), red cell distribution width (RDW), body weight (BW) and murine chemokine CXCL1 (KC).

FIG. 9 illustrates Spearman's rank-order correlation values and the corresponding p-values (in parentheses) for dFI with lifespan. Analysis is shown for two cohorts: Cohort 1 includes all animals with mortality data, Cohort 2 includes the subset of animals from Cohort 1 for which IGF1 measurements were available. Significant correlations (p<0.05) are highlighted in bold.

FIGS. 10A and 10B illustrate total flux (TF) in log scale representing p16-dependent luciferase reporter activity as a quantitative indicator of senescent cells: statistically significant correlations with age (FIG. 10A) and with dFI (FIG. 10B) in old mice (>50 weeks).

FIGS. 11A-11D illustrate dFI responds to the lifespan-modifying effect of high-fat diet (HFD) feeding. (FIGS. 11A and 11C) Kaplan-Meier survival curves showing that long-term (26 weeks) HFD feeding significantly reduces the lifespan of male (FIG. 11A) (p=0.025, log rank test), but not female (FIG. 11C) (p=0.6, log rank test) mice in comparison with regular diet (RD) feeding. (FIGS. 11B and 11D) dFI values measured late in life (at week 78) for male (FIG. 11B) and female (FIG. 11D) mice fed with RD or HFD. Individual animals are represented by dots, with the horizontal bar indicating the group mean value. The horizontal dashed line shows the mean value for animals from both groups. dFI was significantly higher in males with HFD vs RD (p=0.05, Student's t-test), but there was no significant difference between HFD and RD groups of female mice.

FIGS. 12A-12C illustrate effects of 8 week-long rapamycin treatment on body weight and dFI. Body weight (FIG. 12A) and dFI (FIG. 12B) were measured every one and every two weeks, respectively. All of the data are presented as mean±SEM (n=12 mice/group). FIG. 12C illustrates change of dFI between two consecutive measurements when no treatment was given (blue box, includes both control and rapamycin group after withdrawal) or treatment was given (orange box, rapamycin group during treatment period). The change in dFI was significantly lower under treatment (p=0.02, Student's two-tailed t-test).

FIG. 13 illustrates network architecture of nonlinear auto-encoder (AE) with the embedded modes identication. The network is composed of AE, projector, linear dynamics and auxiliary decoder blocks. The AE block encodes input of 12 CBC parameters to the 4dimensional vector and then reconstructs back the original input. The AE consists of fully connected dense layers and residual network blocks (ResNet), which adds nonlinear rectication transformations. The AE is trained simultaneously on cross-sectional and longitudinal datasets. The projector block takes 4-dimensional vector as an input and transforms it to a scalar z, which we refer to as dFI. During training, a pair of vectors is fed to the inputs: one yₙ for the present state of the system and one yₙ+1 for the future state. The linear dynamics block solves the equation of rst-order autoregressive processes and predicts the future state zₙ+1. The auxiliary decoder block reconstructs the original 12-dimension CBC vector from the output of the linear dynamics block utilizing the decoder from the AE block.

FIG. 14 illustrates a schematic of certain residual network (ResNet) blocks. These consist of two fully connected dense layers with activation function of recited linear unit (ReLU). Input and output are interconnected by applying element-wise addition.

FIG. 15 is a table listing all abbreviations used in this disclosure.

FIG. 16 is a table comprising a complete list of datasets used for training models disclosed herein.

FIG. 17 is a table describing the various test datasets used.

FIG. 18 is a table illustrating reconstruction error (root-mean square error, RMSE) and coefficient of determination, R², of the autoencoder calculated for each CBC feature in the training set.

FIG. 19 is a table illustrating reconstruction error (root-mean square error, RMSE) and coefficient of determination, R², of the autoencoder calculated for each CBC feature in the test set.

FIG. 20 illustrates principal component analysis (PCA) of the MPD data (including young animals). The graphs represent the average of the PC scores in subsequent age groups. The inset shows that the variance for all PC scores increase with age.

FIG. 21 is a graph illustrating the growth of age cohort average dFI with age in the training dataset.

FIG. 22 illustrates clustering of CBC features and dFI score in the test dataset. The colors represent the Pearson's correlation coefficient (absolute value) as indicated by the scale on the right side of the figure.

FIG. 23 illustrate correlations between dFI and other biological markers. The colors represent the following datasets: blue represents females in MA0071, orange represents males in MA0071, and green represents males in MA0072.

FIG. 24 illustrates correlations between dFI and CBC parameters in the Peters4 dataset. Colors from blue to red represent age of animals, where blue is age of 26 weeks and red is age of 104 weeks.

FIGS. 25A and 25B illustrate correlations between dFI and CBC parameters in the Peters4 dataset shown for cohort of mice of same strain and sex.

### DETAILED DESCRIPTION

The identification of genes and interventions that slow or reverse aging and treat many aging related conditions is hampered by the lack of metrics that can predict life expectancy of pre-clinical models.

Frailty Indices (FIs) in mice are composite measures of health that are cost-effective and non-invasive, but whether they can accurately predict health and lifespan is not known. Moreover, many of them demand a lot of manual work.

We suggest methods of biological aging determination that are useful for screening anti-aging interventions, evaluating long term effects of any interventions, as pro-longevity and anti-longevity (aka chronic toxicity).

Biomarkers of human aging are also urgently needed for a variety of reasons. These include the identification of individuals at high risk of developing age-associated disease or disability. This would then prompt targeted follow-up examinations and, if available, prophylactic intervention or early-stage treatment of age-related disease. Furthermore, the availability of powerful biomarkers would allow the assessment of the efficacy of forthcoming pharmacological and other interventions (including optimization of micronutrient intake and other dietary components or physical activity) currently being developed and aimed to lower the risk of age-associated disease even in individuals without accelerated aging.

In view of the rapidly increasing average life expectancy of human beings world-wide, the prevalence of age-related diseases is likely to increase as well. This necessitates effective new strategies for prevention and early diagnosis of such conditions as well as for design of treatments. Cost-effective animal models for anti-aging treatment and system for its analysis are needed.

Accordingly, the technical problem underlying the present invention is to provide a method for the determination of the biological age of a mammal.

In some embodiments, the methods of this invention should be applicable to humans in the middle age range (e.g., 30 to 80 years) and should serve as a valuable diagnostic tool for preventive medicine by enabling identification of healthy persons whose aging process is accelerated and who thus are likely to be affected by typical age-related diseases at relatively young chronological age. The solution to the above technical problem is achieved by the embodiments characterized in the claims.

In some of embodiments, the invention provides methods and systems for screening interventions to evaluate its potential to be an anti-aging or geroprotective treatments.

Anti-aging treatment includes (but is not limited to) treatments leading to prevention, amelioration or lessening the effects of aging, decreasing or delaying an increase in the biological age, slowing rate of aging; treatment, prevention, amelioration and lessening the effects of frailty or at least one of aging related diseases and conditions or declines or slowing down the progression of such decline (including but not limited to those indicated in Table 1, “Declines”), condition or disease, increasing health span or lifespan, rejuvenation, increasing stress resistance or resilience, increasing rate or other enhancement of recovery after surgery, radiotherapy, disease and/or any other stress, prevention and/or the treatment of menopausal syndrome, restoring reproductive function, eliminating or decrease in spreading of senescent cells, decreasing all-causes or multiple causes of mortality risks or mortality risks related to at least one or at least two of age related diseases or conditions or delaying in increase of such risks, decreasing morbidity risks. The treatment leading to the modulating at least one of biomarkers of aging into more youthful state or slowing down its change into “elder” state is also regarded to be an anti-aging treatment, including but not limited to biomarkers of aging which are visible signs of aging, such as wrinkles, grey hairs etc. In some embodiments, an age-related disease or disorder is selected from: atherosclerosis, cardiovascular disease, adult cancer, arthritis, cataracts, osteoporosis, type 2 diabetes, hypertension, neurodegeneration (including but not limited to Alzheimer's disease, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence (including but not limited to age related decline in immune response to vaccines, age related decline in response to immunotherapy etc.), myocardial infarction, acute coronary syndrome, sarcopenia, sarcopenic obesity, senile osteoporosis, urinary incontinence etc. Aging-related changes in any parameter or physiological metric are also regarded as age-related conditions, including but not limited to aging related change in blood parameters, heart rate, cognitive functions/decline, bone density, basal metabolic rate, systolic blood pressure, heel bone mineral density (BMD), heel quantitative ultrasound index (QUI), heel broadband ultrasound attenuation, heel broadband ultrasound attenuation, forced expiratory volume in 1-second (FEV1), forced vital capacity (FVC), peak expiratory flow (PEF), duration to first press of snap-button in each round, reaction time, mean time to correctly identify matches, hand grip strength (right and/or left), whole body fat-free mass, leg fat-free mass (right and/or left), and time for recovery after any stress (wound, operation, chemotherapy, disease, change in lifestyle etc.). In some embodiments, the age-related disorder is a cardiovascular disease. In some embodiments, the age-related disorder is a bone loss disorder. In some embodiments, the age-related disorder is a neuromuscular disorder. In some embodiments, the age-related disorder is a neurodegenerative disorder or a cognitive disorder. In some embodiments, the age-related disorder is a metabolic disorder. In some embodiments, the age-related disorder is sarcopenia, osteoarthritis, chronic fatigue syndrome, senile dementia, mild cognitive impairment due to aging, schizophrenia, Huntington's disease, Pick's disease, Creutzfeldt-Jakob disease, stroke, CNS cerebral senility, age-related cognitive decline, pre-diabetes, diabetes, obesity, osteoporosis, coronary artery disease, cerebrovascular disease, heart attack, stroke, peripheral arterial disease, aortic valve disease, stroke, Lewy body disease, amyotrophic lateral sclerosis (ALS), mild cognitive impairment, pre-dementia, dementia, progressive subcortical gliosis, progressive supranuclear palsy, thalamic degeneration syndrome, hereditary aphasia, myoclonus epilepsy, macular degeneration, or cataracts. Aging related change in any parameter of organism is also regarded as an aging related condition, including but not limited to aging related change in at least one of the parameter selected from the Table “Declines”. In some embodiments, term “anti-aging treatment” means treatment increasing resistance to radiation. In some embodiments, term “anti-aging treatment” means treatment against accelerated aging, including but not limited to accelerated aging/frailty after chemotherapy, accelerated aging in HIV, schizophrenia and other diseases and conditions. In some embodiments, methods of this invention are for discovery and evaluation of treatments in cancer supportive care.

Table 1 “Declines”. Any one of the preceding items, wherein instead of device of item 1 at least one other device described in this disclosure is used. Any one of the preceding items, wherein instead of method described in such item at least one other method described in this disclosure is used. Any one of the preceding items, wherein instead of kit described in such item at least one other kit described in this disclosure is used.

TABLE 1
DECLINES

| Field | Units |
|---|---|
| Standing height | cm |
| Forced expiratory volume in 1-second (FEV1) | litres |
| Leg fat-free mass (right) | Kg |
| Leg predicted mass (right) | Kg |
| Basal metabolic rate | KJ |
| Forced vital capacity (FVC) | litres |
| Leg fat-free mass (left) | Kg |
| Leg predicted mass (left) | Kg |
| Systolic blood pressure, automated reading | mmHg |
| Heel bone mineral density (BMD) (left) | g/cm2 |
| Heel quantitative ultrasound index (QUI), direct entry (left) | |
| Whole body fat-free mass | Kg |
| Whole body water mass | Kg |
| Heel bone mineral density (BMD) T-score, | Std. Devs |
| automated (left) | |
| Speed of sound through heel (left) | m/s |
| Sitting height | cm |
| Heel bone mineral density (BMD) (right) | g/cm2 |
| Heel quantitative ultrasound index (QUI), direct entry (right) | |
| Speed of sound through heel (right) | m/s |
| Heel bone mineral density (BMD) T-score, | Std. Devs |
| automated (right) | |
| Peak expiratory flow (PEF) | litres/min |
| Leg fat percentage (left) | percent |
| Trunk fat-free mass | Kg |
| Leg fat percentage (right) | percent |
| Trunk predicted mass | Kg |
| Hand grip strength (left) | Kg |
| Heel broadband ultrasound attenuation (left) | dB/MHz |
| Heel broadband ultrasound attenuation (right) | dB/MHz |
| Hand grip strength (right) | Kg |
| Duration to first press of snap-button in each | milliseconds |
| round | |
| Mean time to correctly identify matches | milliseconds |
| Body fat percentage | percent |
| Trunk fat percentage | percent |
| Body mass index (BMI) | Kg/m2 |
| Leg fat mass (left) | Kg |
| Arm fat-free mass (left) | Kg |
| Arm predicted mass (left) | Kg |
| Arm fat-free mass (right) | Kg |
| Haematocrit percentage | percent |
| Arm predicted mass (right) | Kg |
| Waist circumference | cm |
| Leg fat mass (right) | Kg |
| Haemoglobin concentration | grams/decilitre |
| Arm fat percentage (left) | percent |
| Ankle spacing width (left) | mm |
| Whole body fat mass | Kg |
| Body mass index (BMI) | Kg/m2 |
| Pulse wave peak to peak time | milliseconds |
| Arm fat percentage (right) | percent |
| Weight | Kg |
| Mean corpuscular volume | femtolitres |
| Trunk fat mass | Kg |
| Pulse wave Arterial Stiffness index | |
| Ankle spacing width (right) | mm |
| Platelet crit | percent |
| Red blood cell (erythrocyte) count | 10\hat{ }12 |
| | cells/Litre |
| Mean sphered cell volume | femtolitres |
| Mean platelet (thrombocyte) volume | femtolitres |
| Weight | Kg |
| Arm fat mass (left) | Kg |
| Lymphocyte percentage | percent |
| Neutrophill percentage | percent |
| Arm fat mass (right) | Kg |
| Impedance of leg (left) | ohms |
| Mean reticulocyte volume | femtolitres |
| Platelet count | 10\hat{ }9 |
| | cells/Litre |
| Mean corpuscular haemoglobin | picograms |
| Impedance of leg (right) | ohms |
| Red blood cell (erythrocyte) distribution width | percent |
| Pulse rate, automated reading | bpm |
| Impedance of whole body | ohms |
| Diastolic blood pressure, automated reading | mmHg |
| Lymphocyte count | 10\hat{ }9 |
| | cells/Litre |
| Number of measurements made | 10\hat{ }9 |
| Neutrophill count | cells/Litre |
| Monocyte percentage | percent |
| Hip circumference | cm |
| Monocyte count | 10\hat{ }9 |
| | cells/Litre |
| Platelet distribution width | percent |
| Mean corpuscular haemoglobin concentration | grams/decilitre |
| Immature reticulocyte fraction | ratio |
| Impedance of arm (right) | ohms |
| Reticulocyte percentage | percent |
| Number of times snap-button pressed | |
| White blood cell (leukocyte) count | 10\hat{ }9 |
| Pulse rate | bpm |
| High light scatter reticulocyte count | 10\hat{ }12 |
| | cells/Litre |
| Basophill percentage | percent |
| Impedance of arm (left) | ohms |
| Pulse wave reflection index | |
| Eosinophill count | 10\hat{ }9 |
| | cells/Litre |
| Nucleated red blood cell count | 10\hat{ }9 |
| | cells/Litre |
| Eosinophill percentage | percent |
| | 10\hat{ }9 |
| Basophill count | cells/Litre |
| | 10\hat{ }12 |
| Reticulocyte count | cells/Litre |
| High light scatter reticulocyte percentage | percent |
| Nucleated red blood cell percentage | percent |

Non-limiting list of parameters which age related change is regarded as age related decline and which can be changed into younger state or stabilized or its further change into the older state delayed by anti-aging intervention discovered with the use of methods of this invention.

In some embodiments, the biological age is understood as the distance measured along a continuous trajectory consisting of distinct phases, each corresponding to subsequent human life stages as described in more details in “Quantitative Characterization of Biological Age and Frailty Based on Locomotor Activity Records”, Pyrkov et al., 2017) https://www.biorxiv.org/content/biorxiv/early/2017/09/09/186569.full.pdf.

In some embodiments, the biological age is understood in the following context. The confinement of the aging dynamics of the physiological variables to the low-dimensional manifold representing the aging trajectory is a hallmark of criticality. It has been long suggested that the regulatory systems governing the dynamics of the organism state vector operate near the order-disorder boundary. The biological age is then the order parameter, associated with the organism development and aging, satisfies a stochastic Langevin equation in an unstable effective potential characterize by the single number, the underlying regulatory network stiffness. The number describes the organism state deviations from the youthful state and has the meaning of the number of regulatory abnormalities accumulated over the course of the organism life history, is associated with the decreased resilience and amplified risks of morbidities and death. stochastic biological age dynamics is the mechanistic origin of Gompertz mortality law. The exponential acceleration of the morbidity and mortality rates is the characteristic feature of aging in adult individuals or older. The reduction of the aging dynamics to essentially a one-dimensional manifold, a consequence of the criticality of the underlying regulatory network, means that the distance traveled along the aging trajectory is thus a progress indicator of the process of aging and hence is a natural biomarker of age. The biological age acceleration, i.e., the difference between the biological age of an individual and average the biological age prediction in the sex- and the age-matched cohort of their peers, is elevated for patients with chronic diseases. It is a powerful predictor of all-cause mortality even after confounding by the standard Health Risks Assessment (HRA) variables such as age, sex, and smoking status.

In some embodiments, for humans, the biological age is understood as the biomarker or metric based on one or more several biomarkers predicting risks of morbidity and/or death in 8 years or later or in range of mortality rate doubling time or later.

In some embodiments, for mammals, the biological age is understood as the biomarker or metric based on one or more several biomarkers predicting risks of morbidity and/or death in range of mortality rate doubling time or later.

In some embodiments, the algorithm for biological age determination can be built using machine learning technics, including but not limited to:

1. Supervised Learning

This algorithm consist of a target/outcome variable (or dependent variable) which is to be predicted from a given set of predictors (independent variables). Using these set of variables, we generate a function that map inputs to desired outputs. The training process continues until the model achieves a desired level of accuracy on the training data. Non-limiting Examples of Supervised Learning: Regression, Decision Tree, Random Forest, KNN, Logistic Regression etc.

2. Unsupervised Learning

In this algorithm, we do not have any target or outcome variable to predict/estimate. It is used for clustering population in different groups, which is widely used for segmenting customers in different groups for specific intervention. Examples of Unsupervised Learning: Apriori algorithm, K-means.

3. Reinforcement Learning

Using this algorithm, the machine is trained to make specific decisions. It works this way: the machine is exposed to an environment where it trains itself continually using trial and error. This machine learns from past experience and tries to capture the best possible knowledge to make accurate business decisions.

Example of Reinforcement Learning: Markov Decision Process

Non-Limiting List of Common Machine Learning Algorithms

Here is a list of commonly used machine learning algorithms. These algorithms can be applied to almost any data problem disclosed herein:
Linear Regression
Logistic Regression
Decision Tree
SVM
Naive Bayes
kNN
K-Means
Random Forest
Dimensionality Reduction Algorithms
Gradient Boosting algorithms
GBM
XGBoost
LightGBM
CatBoost

In some embodiments, such machine learning technics can be used to build algorithm of biological age determination as disclosed herein:
Artificial neural network
Random Forests
Ensembles of classifiers
Bootstrap aggregating
Decision tree
Linear classifier
Linear regression
Logistic regression
Support vector machine
Canonical correlation analysis
Factor analysis
Principal component analysis
Partial least squares regression
Principal component regression

In some embodiments, the computer implemented method of this invention is implemented in the form of a python script.

The implementation can be as a computer program product, e.g., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be recorded in any form of programming language, including compiled or interpreted languages, and the computer program can be deployed in any form, including as a stand-alone program or as a subroutine, element, or other unit suitable for use in a computing environment. A computer program can be deployed to be executed on one computer or on multiple computers at one site or several sites.

In some embodiments, any method of this invention, including but not limited to method described in “Items” can be performed by one or more programmable processors executing a computer program to perform functions of the invention by operating on input data and generating output. It can also be performed by, and an apparatus can be implemented as, special purpose logic circuitry, e.g., an FPGA (field programmable gate array) or an ASIC (application-specific integrated circuit). Subroutines can refer to portions of the computer program and/or the processor/special circuitry that implements that functionality.

Processors suitable for the execution of a computer program related to this invention include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer also includes, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

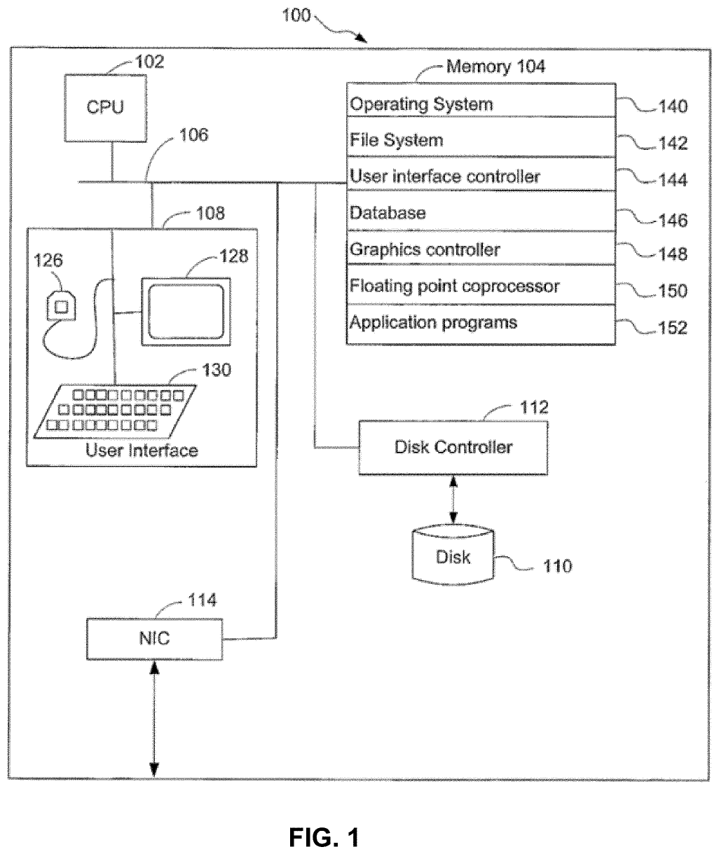

FIG. 1 shows a schematic of a general-purpose computer system 100 suitable for practicing the methods described herein. The computer system 100, shown as a self-contained unit but not necessarily so limited, comprises at least one data processing unit (CPU) 102, a memory 104, which will typically include both high speed random access memory as well as non-volatile memory (such as one or more magnetic disk drives) but may be simply flash memory, a user interface 108, optionally a disk 110 controlled by a disk controller 112, and at least one optional network or other communication interface card 114 for communicating with other computers as well as other devices. At least the CPU 102, memory 104, user interface 108, disk controller where present, and network interface card, communicate with one another via at least one communication bus 106.

Memory 104 stores procedures and data, typically including: an operating system 140 for providing basic system services; application programs 152 such as user level programs for viewing and manipulating data, evaluating formulae for the purpose of diagnosing a test subject; authoring tools for assisting with the writing of computer programs; a file system 142, a user interface controller 144 for handling communications with a user via user interface 108, and optionally one or more databases 146 for storing microarray data and other information, optionally a graphics controller 148 for controlling display of data, and optionally a floating point coprocessor 150 dedicated to carrying out mathematical operations. The methods of the present invention may also draw upon functions contained in one or more dynamically linked libraries, not shown in FIG. 1, but stored either in Memory 104, or on disk 110, or accessible via network interface connection 114.

User interface 108 may comprise a display 128, a mouse 126, and a keyboard 130. Although shown as separate components in FIG. 1, one or more of these user interface components can be integrated with one another in embodiments such as handheld computers. Display 128 may be a cathode ray tube (CRT), or flat-screen display such as an LCD based on active matrix or TFT embodiments, or may be an electroluminescent display, based on light emitting organic molecules such as conjugated small molecules or polymers. Other embodiments of a user interface not shown in FIG. 1 include, e.g., several buttons on a keypad, a card-reader, a touch-screen with or without a dedicated touching device, a trackpad, a trackball, or a microphone used in conjunction with voice-recognition software, or any combination thereof, or a security-device such as a fingerprint sensor or a retinal scanner that prohibits an unauthorized user from accessing data and programs stored in system 100. System 100 may also be connected to an output device such as a printer (not shown), either directly through a dedicated printer cable connected to a serial or USB port, or wirelessly, or via a network connection.

The database 146 may instead, optionally, be stored on disk 110 in circumstances where the amount of data in the database is too great to be efficiently stored in memory 104. The database may also instead, or in part, be stored on one or more remote computers that communicate with computer system 100 through network interface connection 114.

The network interface 134 may be a connection to the internet or to a local area network via a cable and modem, or ethernet, firewire, or USB connectivity, or a digital subscriber line. Preferably the computer network connection is wireless, e.g., utilizing CDMA, GSM, or GPRS, or Bluetooth, or standards such as 802.11a, 802.11b, or 802.11g.

It would be understood that various embodiments and configurations and distributions of the components of system 100 across different devices and locations are consistent with practice of the methods described herein. For example, a user may use a handheld embodiment that accepts data from a test subject, and transmits that data across a network connection to another device or location wherein the data is analyzed according to a formulae described herein. A result of such an analysis can be stored at the other location and/or additionally transmitted back to the handheld embodiment. In such a configuration, the act of accepting data from a test subject can include the act of a user inputting the information. The network connection can include a web-based interface to a remote site at, for example, a lab researcher or healthcare provider. Alternatively, system 100 can be a device such as a handheld device that accepts data from the test subject, analyzes the data, such as by inputting the data into a formula as further described herein, and generating a result that is displayed to the user. The result can then be, optionally, transmitted back to a remote location via a network interface such as a wireless interface. System 100 may further be configured to permit a user to transmit by e-mail results of an analysis directly to some other party, such as a researcher, customer, healthcare provider, or a diagnostic facility, or a patient

In some embodiments, Neural network was implemented using python 3 and tensorflow framework.

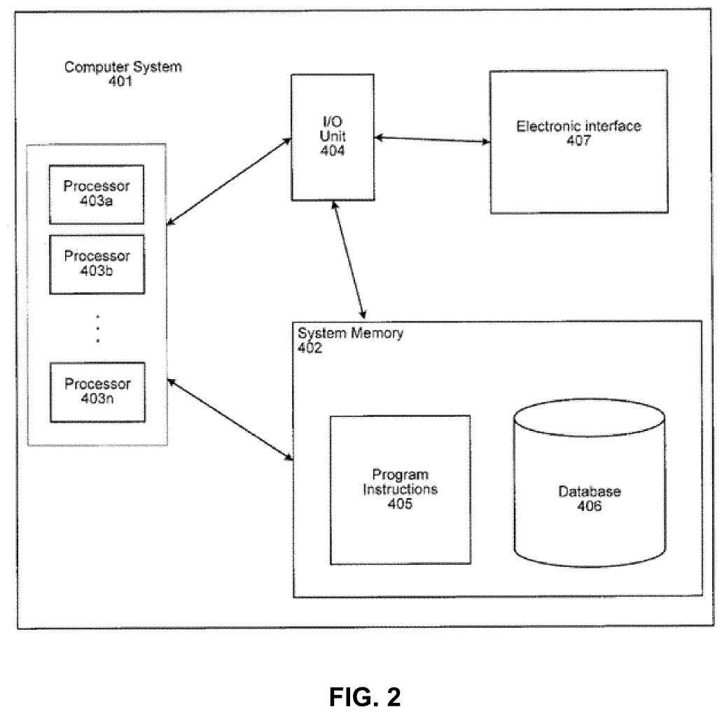

FIG. 2 is a block diagram that illustrates an exemplary computer system in accordance with one or more embodiments of the present invention.

Exemplary embodiments of the present invention include an online biological age determination system, as illustrated by using an example in FIG. 2. An online system indicates that the system is accessible to a user over a network and may encompass accessibility through data networks, including but not limited to the internet, intranets, private networks or dedicated channels. This online biological age determination system 401 includes one or more processors 403 a-403 n, an input/output unit 404 adapted to be in communication with the one or more processors, one or more databases 406 in communication with the one or more processors to store, use and associate a plurality of values of health parameters, algorithm, biological age values, one or more electronic interfaces 407 positioned to display an online biological age value and defining interfaces, and non-transitory computer-readable medium 402. The non-transitory computer-readable medium is positioned in communication with the one or more processors and has one or more computer programs stored thereon including a set of instructions 405. This set of instructions when executed by one or more processors cause the one or more processors to perform operations of determination of biological age, interface to display to a user thereof one or more values of health parameters and biological age value responsive to receiving the plurality of health parameters values from the one or more databases or input devices and outputting to the one or more electronic interfaces 407 the online biological age representation. The interface allows an input of a plurality of values of health parameters associated with a mammal.

In certain embodiments, the set of instructions may further include determining biological age for the group of mammals. Various portions of systems and methods described herein, may include or be executed on one or more computer systems similar to system 401.

In some embodiments, the biological age determination system includes one or more processors, an input/output unit adapted to be in communication with the one or more processors, one or more databases in communication with the one or more processors to store and associate a plurality of values of heath parameters with a plurality of biological age values; and non-transitory computer-readable medium. This non-transitory computer-readable medium is positioned in communication with the one or more processors and having one or more computer programs stored thereon including a set of instructions.

The processor can be any commercially available terminal processor, or plurality of terminal processors, adapted for use in or with the computer 41 or system 401. A processor may be any suitable processor capable of executing/performing instructions. A processor may include a central processing unit (CPU) that carries out program instructions to perform the basic arithmetical, logical, and input/output operations of the computer 41 or system 401. A processor may include code (e.g., processor firmware, a protocol stack, a database management system, an operating system, or a combination thereof) that creates an execution environment for program instructions. A processor may include a programmable processor. A processor may include general and/or special purpose microprocessors. The processor can be, for example, the Intel® Xeon® multicore terminal processors, Intel® micro-architecture Nehalem, and AMD Opteron™ multicore terminal processors, Intel® Core® multicore processors, Intel® Core iSeries® multicore processors, and other processors with single or multiple cores as is known and understood by those skilled in the art. The processor can be operated by operating system software installed on memory, such as Windows Vista, Windows NT, Windows XP, UNIX or UNIX-like family of systems, including BSD and GNU/Linux, and Mac OS X. The processor can also be, for example the TI OMAP 3430, Arm Cortex A8, Samsung S5PC100, or Apple A4. The operating system for the processor can further be, for example, the Symbian OS, Apple iOS, Blackberry OS, Android, Microsoft Windows CE, Microsoft Phone 7, or PalmOS. Computer system 401 may be a uni-processor system including one processor (e.g., processor 403 a), or a multi-processor system including any number of suitable processors (e.g., 403 a-403 n). Multiple processors may be employed to provide for parallel and/or sequential execution of one or more portions of the techniques described herein. Processes and logic flows described herein may be performed by one or more programmable processors executing one or more computer programs to perform functions by operating on input data and generating corresponding output. Processes and logic flows described herein may be performed by, and apparatus can also be implemented as, special purpose logic circuitry, e.g., an FPGA (field programmable gate array) or an ASIC (application specific integrated circuit). Computer system 1000 may include a computer system employing a plurality of computer systems (e.g., distributed computer systems) to implement various processing functions.

A computer 401 as illustrated in the example described in FIG. 2 can further include a non-transitory memory or more than one non-transitory memories (referred to as memory 402 herein). Memory 402 can be configured, for example, to store data, including computer program product or products, which include instructions for execution on the processor. Memory can include, for example, both non-volatile memory, e.g., hard disks, flash memory, optical disks, and the like, and volatile memory, e.g., SRAM, DRAM, and SDRAM as required to support embodiments of the instant invention. As one skilled in the art will appreciate, though the memory 402 is depicted on, e.g., a motherboard, of the computer 401, the memory 402 can also be a separate component or device, e.g., flash memory, connected to the computer 401 through an input/output unit or a transceiver. As one skilled in the art will understand, the program product or products, along with one or more databases, data libraries, data tables, data fields, or other data records can be stored either in memory 402 or in separate memory (also non-transitory), for example, associated with a storage medium such as a database (not pictured) locally accessible to the computer 401, positioned in communication with the computer 401 through the I/O device. Non-transitory memory further can include drivers, modules, libraries, or engines allowing the genetic merit scorecard computer to function as a dedicated software/hardware system (i.e., a software service running on a dedicated computer) such as an application server, web server, database server, file server, home server, standalone server. For example, non-transitory memory can include a server-side markup language processor (e.g., a PHP processor) to interpret server-side markup language and generate dynamic web content (e.g., a web page document) to serve to client devices over a communications network.

Embodiments of the present invention include generating a interface for acquiring the information associated with the mammals, for example, values of health parameters, such as but not limited to results of CBC blood tests, mammals IDs, management information, and other information relevant to the assessment of the biological age. In an exemplary embodiment of the present invention, the interface is generated by a computer program product in communication with a computer associated with a biological age determination system. As used herein, an interface can a graphical user interface facilitating the acquisition of data from the user to determine the biological age of an animal or a plurality of animals. This electronic interface can also display the genetic merit scorecard. The graphical user interface device can include, for example, a CRT monitor, a LCD monitor, a LED monitor, a plasma monitor, an OLED screen, a television, a DLP monitor, a video projection, a three-dimensional projection, a holograph, a touch screen, or any other type of user interface which allows a user to interact with one of the plurality of remote computers using images as is known and understood by those skilled in the art.

In some embodiments, one or more of the biological age estimations can be outputted via one or more data communication protocols well known in the art, including, but not limited to, Wi-Fi, Bluetooth, I2C, UART, USB, Ethernet, TCP/IP, Remote Procedure Calls (RPCs), or custom-designed data transmitting protocols over wired or wireless channels. Such embodiments may be part of a larger system. For example, the embodiment may be embedded into a computer or smart apparel or smartphone for enhanced data processing and storage power or may be used as part of a health monitoring system.

### EXAMPLES

To screen compounds for potential anti-aging or toxicity effects the mice should be administered in therapeutically effective amount in a manner.

1. Rapamycin is administered at 12 mg/lg via oral gavage for 12 weeks to C57BL/6J male mice aged 60 weeks (Jackson Laboratories, USA), 12 animals per group, control group with vehicle.

2. After 4 weeks of treatment, a standard blood count analysis should be performed and estimated the biological age.

3. Biological age reduction is detected after 4 weeks of treatment in rapamycin treatment groups compared to vehicle control. The example of biological calculation is presented in the section titled “Identification of a Blood Test-Based Biomarker of Aging through Deep Learning of Aging Trajectories in Large Phenotypic Datasets of Mice.”

In some of the embodiments, The bio age calculation procedure consists of the following stages:

1) subtract the reference mean value (column MEAN in the table) of each test;

2) multiply by the coefficient from column COEF;
MEAN,COEF
HB (g/dL),14.7810810811,−0.324994418476
LY (K/uL),6.78821787942,−0.0403357974256
MCH (Pg),15.2156964657,−0.305640352983
MCHC (g/dL),33.18497921,0.0243410007583 MCV(fL),45.8556652807,−0.071912079313
MO (K/uL),0.187391325364,2.99337099222 MPV,5.82976611227,−0.0622717180147
PLT,1258.6456341,0.00122980926892
RBC (M/uL),9.74016632017,−0.227470069201
WBC (K/uL),8.83614345114,0.0437124309324 3) sum the resulting values.

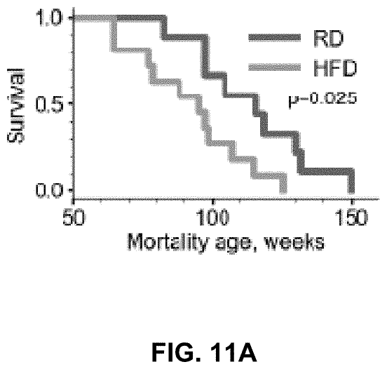

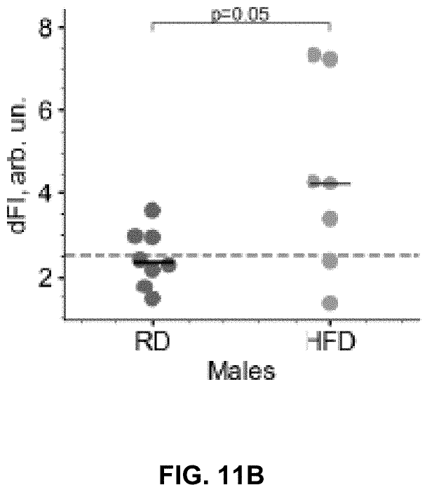

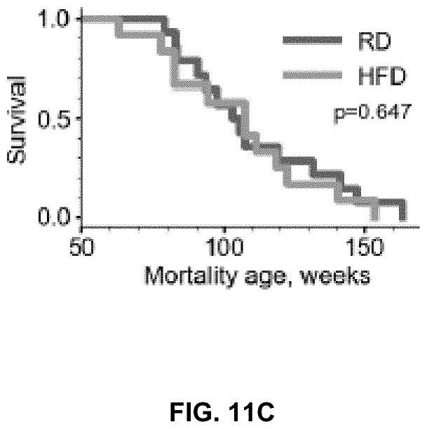

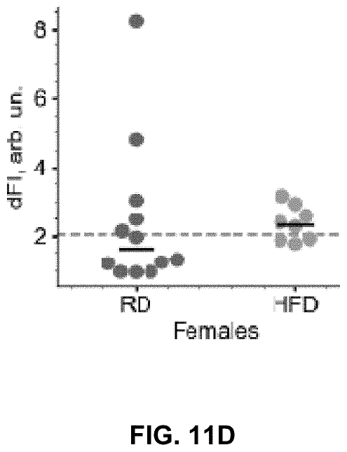

FIGS. 11A-11D illustrates evidence for algorithm for biological age determination efficacy in sensing anti-longevity or toxic interventions such as high fat diet, wherein blood from mice was obtained in about 20 weeks after start of high fat diet.

A larger biological age value, therefore, corresponds to a shorter lifespan and the other way around. The reduction of biological age would imply that the animal is rejuvenated to some extend and health span and lifespan expectancy is increased. Therefore, the intervention that lead to this effect is expected to have an anti-aging treatment potential.

### Example

How it was Done in Mice

NN model was trained using the best overlap of available CBC features from all sources. The final list contained 12 CBC features: granulocytes differential (gr, %), granulocytes count (gr, K/μl), hemoglobin (hb, g/dl), hematocrit (hct, %), lymphocyte differential (ly, %), lymphocyte count (ly, K/μl), mean corpuscular hemoglobin content (mch, pg), mean hemoglobin concentration (mchc, g/dl), mean corpuscular volume (mcv, fl), platelet count (plt, K/μl), red blood cell count (rbc, M/μl) and white blood cell count (wbc, K/μl).

No matter how the NN was trained, the architecture of NN or values of weights used in NN layers, the score (or biological age) should have the following property: the correlation coefficient between values of the score at any time point and its value with a time lag Δt>10 weeks should be higher than 0.5. On FIG. 1 we show correlation between scores of male mice at the age range 66-110 weeks. To calculate correlation one should take values of the score for each mice and form a vector X, then take values of the score for the same mice but calculated in the next time point with a lag Δt and form the vector Y (the ordering of mice corresponds to the ordering in vector X). Finally, we compute the Pearson correlation coefficient between vectors X and Y. For example, the correlation coefficient between scores measured at the time lag of 14 weeks is 0.58, and at the time lag of 28 weeks is 0.66.

In some embodiments, We claim that our invention covers any score used for calculation of biological age with any computer algorithms with correlation coefficient higher than threshold value of 0.5 using our benchmark dataset. The benchmark dataset contains 12 CBC features for male mice measured at 66, 81, 94, 109 and 130 weeks.

Accordingly, the present invention also relates to the following items:

Items

Item 1: Method for determining the biological age of mammals, the method comprising:
inputting values of at least six health parameters from a mammal; and
determining the biological age of the mammal by calculating the biological age of the mammal using an algorithm comprising multiple mathematical operations, wherein the algorithm is defined by a Pearson correlation coefficient higher than 0.5, wherein the Pearson correlation coefficient is determined by:
   a. calculating a first biological age of a plurality of mammals of the same phenotype at a first time represented by a first vector X;
   b. calculating a second biological age of the plurality of mammals of the same phenotype at a second time represented by a second vector Y; and
   c. determining the Pearson correlation coefficient between vectors X and Y.

Item 2: At least one of the methods for determining the biological age of mammals, selected from the methods described in the section titled “Identification of a Blood Test-Based Biomarker of Aging through Deep Learning of Aging Trajectories in Large Phenotypic Datasets of Mice.”

Item 3: At least one of the methods for training a model for determining the biological age of mammals, selected from the methods described in the section titled “Identification of a Blood Test-Based Biomarker of Aging through Deep Learning of Aging Trajectories in Large Phenotypic Datasets of Mice.”

Item 4: At least one of the methods for building a model for determining the biological age of mammals, selected from the methods described in the section titled “Identification of a Blood Test-Based Biomarker of Aging through Deep Learning of Aging Trajectories in Large Phenotypic Datasets of Mice.”

Item 5: Method for determining the biological age of a mammal, the method comprising:
inputting values of at least six of health parameters of the mammals into computer,
a calculation of biological age by application of algorithm comprising performance of multiple mathematical operations, at least multiplication by matrix and summation of vectors to inputted values of health parameters (those values of health parameters that were inputted according to the previous step), wherein said biological age is a single number (score), and the said algorithm has at least the following features:
a. if one will use the said algorithm to determine scores using values of the same health parameters of at least 50 of mammals of the same phenotype, wherein each individual animal must have a unique identification label (e.g., A1 for animal 1, A2 for animal 2 etc.),
b. repeat clause (a) with the same mammals but health parameters are obtained from the same individual animals not later than period of 10% of such mammals' average lifespan after the date of obtaining health parameters from the same individual animal in clause (a)
c. Than a Pearson correlation coefficient between vectors X and Y will have value higher than of 0.5, if Pearson correlation calculated in the following way: one should take values of the score for each animal from clause (a) and form a vector X, then take values of the score from clause (b) and form the vector Y, wherein to construct both vectors X and Y the scores should be placed to keep ordering of identification labels (e.g., X=[scoret1a1, scoret1a2, . . . , scoret1a50) and Y=[scoret2a1, scoret2a2, scoret2a50)

Item 6: Method of any one of preceding items, wherein Pearson correlation coefficient is selected from the group: higher than of 0.55, higher than of 0.6, in the range from 0.5 to 0.7, in the range from 0.6 to 0.8, in the range from 0.5 to 0.9, in the range from 0.5 to 0.99, in the range from 0.55 to 0.99, higher than of 0.7, higher than of 0.8, higher than of 0.9, higher than of 0.95, higher than of 0.99.

Item 7: Method for determining the biological age of mammals, the method comprising: inputting values of at least six health parameters from a mammal; and determining the biological age of the mammal by calculating the biological age of the mammal using an algorithm comprising multiple mathematical operations, wherein the algorithm predicts scores which order animals by their survival time, where in the spearman's rank-order correlation between such scores and real survival times should be negative number with the corresponding p-values lower than 0.05.

Item 8: Method of any one of preceding items, wherein spearman's rank-order correlation p-values 1 is selected from the following group: lower than 0.03, lower than 0.01, lower than 0.005, lower than 0.003, lower than 0.001, lower than 0.0005, lower than 0.0003, lower than 0.0001, lower than 0.00005, lower than 0.00003, lower than 0.00001, lower than 0.000001, lower than 0.0000001, in the range from 0.05 to 0.0000001, in the range from 0.01 to 0.000001, in the range from 0.001 to 0.00001.

Item 9: Method of any one of preceding items, wherein p-value is selected from the following group for a corresponding number of mammals:
N p-value
for 20 mammals—lower than 0.05,
for 20 mammals—lower than 0.03,
for 20 mammals—lower than 0.01,
for 20 mammals—in the range from 0.04 to 0.01,
for 20 mammals—in the range from 0.04 to 0.001,
for 30 mammals—lower than 0.02,
for 50 mammals—lower than 0.01,
for 50 mammals—lower than 0.001,
for 100 mammals—lower than 0.001
for 150 mammals—lower than 1E-05,
   for >200 mammals—lower than 1E-6.

Item 10: Method of any one of preceding items, wherein the biological age is a score.

Item 11: Method of any one of preceding items, wherein the biological age is a score preferably a single value.

Item 12: Method of any one of preceding items, wherein the mathematical operations comprise multiplication of matrices and summation of vectors of inputted values of the health parameters.

Item 13: Method of any one of preceding items, wherein of the same phenotype is at least 10 mammals, is at least 25 mammals, is at least 50 mammals, is at least 100 mammals, is at least 500 mammals.

Item 14: Method of any one of preceding items, further comprising determining the algorithm using a neural network architecture.

Item 15: Method any one of preceding items, wherein determining the algorithm comprises: obtaining health parameters and corresponding ages from a plurality of mammals; and inputting the health parameters and the corresponding ages of the mammals into an autoencoder of the neural network architecture.

Item 16: Method of any one of preceding items, wherein the biological age is a score preferably a single number.

Item 17: Method of any one of preceding items, further comprising determining the algorithm using a neural network architecture, created as shown in the section titled “Identification of a Blood Test-Based Biomarker of Aging through Deep Learning of Aging Trajectories in Large Phenotypic Datasets of Mice” under the sub-section titled “Materials and Methods” and the portion titled “Neural Network Structure.”

Item 18: Method of any one of preceding items, wherein the health parameters are determined based on blood parameters.

Item 19: Method of any one of preceding items, wherein the mammals are one of: mice, humans, dogs, cats, non-human primates, rats, guinea pigs, rabbits, hamsters, sheep, gerbils, bats, ferrets, chinchillas, goats, and horses.

Item 20: Method of any one of preceding items, wherein mammal is alive.

Item 21: Method of any one of preceding items, wherein none of the values of health parameter is zero.

Item 22: Method of any one of preceding items, wherein none of the values of health parameter is equal or around the value of such parameter in a dead mammal of such phenotype.

Item 23: Method of any one of preceding items, wherein number of health parameters values is selected from the group: Seven, Eight, Nine, Ten, Eleven, Twelve, Thirteen and Fourteen.

Item 24: Method of any one of preceding items, wherein health parameters are selected from the following blood parameters: granulocytes differential (gr, %), granulocytes count (gr, K/l), hemoglobin (hb, g/dl), hematocrit (hct, %), lymphocyte differential (ly, %), lymphocyte count (ly, K/l), mean corpuscular hemoglobin content (mch, pg), mean hemoglobin concentration (mchc, g/dl), mean corpuscular volume (mcv, fl), platelet count (plt, K/l), red blood cell count (rbc, M/l) and white blood cell count (wbc, K/l).

Item 25: Method of any one of preceding items, wherein health parameters are granulocytes differential (gr, %), granulocytes count (gr, K/l), hemoglobin (hb, g/dl), hematocrit (hct, %), lymphocyte differential (ly, %), lymphocyte count (ly, K/l), mean corpuscular hemoglobin content (mch, pg), mean hemoglobin concentration (mchc, g/dl), mean corpuscular volume (mcv, fl), platelet count (plt, K/l), red blood cell count (rbc, M/l) and white blood cell count (wbc, K/l).

Item 26: Method of any one of preceding items, wherein granulocytes are unavailable, it is calculated using the following formulas:

gr(K/l)=wbc(K/l)−ly(K/l)−mo(K/l)gr(%)=100−ly(%)−mo(%)

Item 27: Method of any one of preceding items, wherein health parameters are selected from Complete Blood Count.

Item 28: Method of any one of preceding items, wherein health parameters are Complete Blood Count.

Item 29: Method of any one of preceding items, wherein health parameters comprise HB (g/dL), LY (K/uL), MCH (Pg), MCHC (g/dL), MCV(fL), MO (K/uL), PLT, RBC (M/uL), WBC (K/uL).

Item 30: Method of any one of preceding items, wherein the determination of biological age comprises following steps:

1) subtract the reference mean value (column MEAN in the table) of each test;
2) multiply by the coefficient from column COEF;

### MEAN,COEF

HB (g/dL),14.7810810811,−0.324994418476

### LY (K/uL),6.78821787942,−0.0403357974256

### MCH (Pg),15.2156964657,−0.305640352983

MCHC (g/dL),33.18497921,0.0243410007583 MCV(fL),45.8556652807,−0.071912079313

### MO (K/uL),0.187391325364,2.99337099222 MPV,5.82976611227,−0.0622717180147

### PLT,1258.6456341,0.00122980926892

### RBC (M/uL),9.74016632017,−0.227470069201

### WBC (K/uL),8.83614345114,0.0437124309324

3) sum the resulting values, wherein the sum will be a biological age.

Item 31: Method of preceding item, wherein at least one of COEF differs from the COEF in preceding item about 0.05%, about 0.01%, about 0.1%, about 0.5%, about 1%, about 3%, about 5%, about 10%, about 20%.

Item 32: Method of any one of preceding items, wherein health parameters are selected from Complete Blood Count, Basic Metabolic Panel, Comprehensive Metabolic Panel, Lipid Panel, Liver Panel, Thyroid Stimulating Hormone, Hemoglobin A1C, c-reactive protein.

Item 33: Method of any one of preceding items, wherein health parameters are selected from the group: Glucose, serum (mg/dL); Creatinine (mg/dL); Lactate dehydrogenase LDH (U/L); Uric acid (mg/dL); Blood lead (ug/dL); Homocysteine(umol/L); Vitamin A (ug/dL); Fasting Glucose (mg/dL); GGT: SI (U/L); Total cholesterol (mg/dL); Vitamin E (ug/dL); Chloride: SI (mmol/L); AST: SI (U/L); or Sodium: SI (mmol/L).

Item 34: Method of any one of preceding items, wherein health parameters are selected from the group: PCB180 (ng/g); Cholesterol (mg/dL); PCB170 (ng/g); Alkaline phosphatase (U/L); PCB180 Lipid Adjusted; Oxychlordane Lipid Adjusted; 3,3′,4,4′,5,5′-hexachlorobiphenyl (hxcb) (fg/g); PCB74 (ng/g); PCB170 Lipid Adjusted; Triglycerides (mg/dL); PCB153 (ng/g); Oxychlordane (ng/g); PCB74 Lipid Adjusted; Monocyte percent (%); Ferritin (ng/mL); 3,3′,4,4′,5,5′-hexachlorobiphenyl (hxcb) Lipid Adjusted; 2,3,4,7,8-Pentachlorodibenzofuran (pncdf) (fg/g); Methylmalonic acid (umol/L); PCB153 Lipid Adjusted; PCB187 (ng/g); 2,3,4,7,8-Pentachlorodibenzofuran (pncdf) Lipid Adjusted; PCB156 (ng/g); White blood cell count: SI; PCB187 Lipid Adjusted; 1,2,3,6,7,8-Hexachlorodibenzo-p-dioxin (hxcdd)(fg/g); Trans-nonachlor Lipid Adjusted; PCB138 (ng/g); 4-pyridoxic acid (nmol/L); Potassium: SI (mmol/L); Trans-nonachlor (ng/g); 1,2,3,6,7,8-Hexachlorodibenzo-p-dioxin (hxcdd) Lipid Adjusted; PCB138 Lipid Adjusted; PCB118 (ng/g); PCB156 Lipid Adjusted; PCB118 Lipid Adjusted; Mean cell volume (fL); PCB146 (ng/g); Blood cadmium (ug/L); Two hour oral glucose tolerance (OGTT) (mg/dL); Folate, serum (ng/mL); PCB194 Lipid Adjusted; PCB194 (ng/g); Hematocrit (%); 1,2,3,4,7,8-Hexachlorodibenzofuran (hcxdf) (fg/g); Perfluorohexane sulfonic acid (ug/L); RBC folate (nmol/L); PCB99 (ng/g); p,p′-DDE (ng/g); p,p′-DDE Lipid Adjusted; Total Serum Foalte (nmol/L); PCB146 Lipid Adjusted; PCB196 Lipid Adjusted; PCB196 (ng/g); 1,2,3,4,6,7,8,9-Octachlorodibenzo-p-dioxin (ocdd) (fg/g); PCB 183 (ng/g); Perfluorooctane sulfonic acid; 3,3′,4,4′,5-Pentachlorobiphenyl (pncb) (fg/g); trans-lycopene (ug/dL); 1,2,3,7,8-Pentachlorodibenzo-p-dioxin (pncdd) (fg/g); 1,2,3,4,6,7,8-Heptachlororodibenzo-p-dioxin (hpcdd) (fg/g); 3,3′,4,4′,5-Pentachlorobiphenyl (pncb) Lipid Adjusted; 1,2,3,4,7,8-Hexachlorodibenzofuran (hcxdf) Lipid Adjusted; 1,2,3,6,7,8-Hexachlorodibenzofuran (hxcdf) (fg/g); PCB99 Lipid Adjusted; Triiodothyronine (T3), free (pg/mL); 1,2,3,4,6,7,8,9-Octachlorodibenzo-p-dioxin (ocdd) Lipid Adjusted; a-Tocopherol (ug/dL); Blood o-Xylene Result; Beta-hexachlorocyclohexane Lipid Adjusted; Plasma glucose: SI (mmol/L); 1,2,3,7,8-Pentachlorodibenzo-p-dioxin (pncdd) Lipid Adjusted; Parathyroid Hormone (Elecys method) pg/mL; Beta-hexachloro-cyclohexane (ng/g); 1,2,3,4,6,7,8-Heptachlororodibenzo-p-dioxin (hpcdd) Lipid Adjusted; PCB105 (ng/g); PCB177 (ng/g); Hemoglobin (g/dL); Heptachlor Epoxide (ng/g); Perfluorooctanoic acid; Heptachlor Epoxide Lipid Adjusted; or 1,2,3,6,7,8-Hexachlorodibenzofuran (hxcdf) Lipid Adjusted.

Item 35: Method of any one of preceding items, wherein health parameters are selected from the group: PCB183 Lipid Adjusted; 2,3,7,8-Tetrachlorodienzo-p-dioxin (tcdd) (fg/g); Vitamin B12, serum (pg/mL); cis-b-carotene (ug/dL); Cotinine (ng/mL); 1,2,3,7,8,9-Hexachlorodibenzo-p-dioxin (hxcdd) (fg/g); Triglyceride (mg/dL); p,p′-DDT (ng/g); Triiodothyronine (T3), total (ng/dL); PCB105 Lipid Adjusted; 1,2,3,4,7,8-Hexachlorodibenzo-p-dioxin (hxcdd)(fg/g); Mean cell hemoglobin (pg); Dieldrin (ng/g); Folate, RBC (ng/mL RBC); Aldrin; trans-b-carotene (ug/dL); Eosinophils percent (%); Endrin; Bone alkaline phosphotase (ug/L); PCB199 Lipid Adjusted; 1,2,3,4,7,8-Hexachlorodibenzo-p-dioxin (hxcdd) Lipid Adjusted; 1,2,3,7,8,9-Hexachlorodibenzo-p-dioxin (hxcdd) Lipid Adjusted; Dieldrin Lipid Adjusted; p,p′-DDT Lipid Adjusted; Segmented neutrophils percent (%); 2,3,7,8-Tetrachlorodienzo-p-dioxin (tcdd) Lipid Adjusted; Retinyl stearate (ug/dL); PCB151 (ng/g); PCB149 (ng/g); Perfluorononanoic acid (ug/L); PCB177 Lipid Adjusted; PCB178 Lipid Adjusted; PCB209 (ng/g); PCB178 (ng/g); 5-Methyl THF (nmol/L); PCB209 Lipid Adjusted (ng/g); C-peptide (nmol/L) in SI units; Platelet count (%) SI; Blood Bromodichloromethane Result; Total iron binding capacity (ug/dL); Red cell distribution width (%); Blood Chloroform Result; Glycidamide (pmoL/G Hb); Testosterone total (ng/dL); Hexachlorobenzene (ng/g); Apolipoprotein (B) (mg/dL); ALT: SI (U/L); 25-hydroxyvitamin D2+D3; PCB206 Lipid Adjusted; Follicle stimulating hormone (mIU/mL); Basophils percent (%); 2-(N-Methyl-perfluorooctane sulfonamido) acetic acid (ug/L); Vitamin B6 (Pyridoxal 5′-phosphate) test results (nmol/L); Pyridoxal 5′-phosphate (nmol/L); total Lycopene (ug/dL); Blood Methyl t-Butyl Ether (MTBE) Result; Helicobacter pylori (ISR); PCB167 Lipid Adjusted; Mirex (ng/g); Luteinizing hormone (mIU/mL); Blood manganese (ug/L); Mean cell hemoglobin concentration (g/dL); PCB128 (ng/g); a-Cryptoxanthin (ug/dL); Thyroxine, free (ng/dL); cis-Lycopene (ug/dL); Thyroid stimulating hormone (uIU/mL); PCB172 Lipid Adjusted; Blood mercury, total (ug/L); Inorganic mercury, blood (ug/L); 2,2′,4,4′,5,5′-hexabromobiphenyl (pg/g); Vitamin C (mg/dL); Blood m-/p-Xylene Result; PCB167 (ng/g); Mercury, methyl (ug/L); Combined Lutein/zeaxanthin (ug/dL); 2,2′,4,4′,5,6′-hexabromodiphenyl ether (pg/g); Folic acid, serum (nmol/L); Acrylamide (pmoL/G Hb); 2,2′,4,4′,5,5′-hexabromobiphenyl lipid adjusted (ng/g); 2,3,4,6,7,8,-Hexchlorodibenzofuran (hxcdf) (fg/g); total b-Carotene (ug/dL); 25-hydroxyvitamin D3 (nmol/L); Perfluoroundecanoic acid (ug/L); Protoporphyrin (ug/dL RBC); PCB206 (ng/g); PCB157 Lipid Adjusted; Phytofluene (ug/dL); Aldrin Lipid Adjusted; epi-25-hydroxyvitamin D3 (nmol/L); PCB172 (ng/g); PCB66 (ng/g); Endrin Lipid Adjusted; a-carotene (ug/dL); Trans 9, trans 12-octadienoic acid (uM); PCB28 (ng/g); Pefluorodecanoic acid (ug/L); Lymphocyte percent (%); Thyroid stimulating hormone (IU/L); 1,2,3,4,6,7,8-Heptachlorodibenzofuran (hpcdf) (fg/g); Hexachlorobenzene Lipid Adjusted; Mirex Lipid Adjusted; Total dust weight (mg); Insulin: SI (pmol/L); Sieved dust weight (mg); Serum Selenium (ug/L); Lutein (ug/dL); Blood Nitromethane (pg/mL); Gamma-hexachlorocyclohexane Lipid Adjusted; Retinyl palmitate (ug/dL); Trans 9-octadecenoic acid (uM); 1,2,3,7,8,9-Hexachlorodibenzofuran (hxcdf) (fg/g); 1,2,3,4,7,8,9-Heptachlorodibenzofuran (Hpcdf) (fg/g); PCB87 (ng/g); and Red cell count SI. In some embodiments, the two or more biomarkers are selected from the group: Glucose, serum (mg/dl); Creatinine (mg/dl); Lactate dehydrogenase LDH (U/L); Uric acid (mg/dl); Blood lead (ug/dl); Homocysteine(umol/L); Vitamin A (ug/dl); Fasting Glucose (mg/dl); GGT: SI (U/L); Total cholesterol (mg/dl); Vitamin E (ug/dl); Chloride: SI (mmol/L); AST: SI (U/L); Sodium: SI (mmol/L); PCB180 (ng/g); Cholesterol (mg/dl); PCB170 (ng/g); Alkaline phosphatase (U/L) and glycohemoglobin, glucose serum, glycohemoglobin, creatine, lactate dehydrogenase, uric acid, blood lead, homocysteine, vitamin A, fasting glucose, gamma glutamyltransferase (GGT), total cholesterol, Vitamin E, chloride, aspartate aminotransferase (AST), sodium, and 2,2′,3,4,4′,5,5′-heptachlorobiphenyl (PCB180), glucose serum, glycohemoglobin, creatine, lactate dehydrogenase, uric acid, blood lead, homocysteine, vitamin A, fasting glucose, gamma glutamyltransferase (GGT), and total cholesterol. In some embodiments, biomarkers characteristic of aging are selected from: glucose serum, glycohemoglobin, creatine, lactate dehydrogenase, uric acid, melatonin and blood lead.

Item 36: Method of any one of preceding items, further comprising a step of obtaining a value of health parameter of mammal, preceding its inputting.

Item 37: Method of any one of preceding items, further comprising a step of obtaining sample from of mammal, preceding obtaining a value of health parameter of such mammal.

Item 38: Method of any one of preceding items, further comprising step of using frailty index to increase the quality of biological age determination.

Item 39: Method of any one of preceding item, wherein the biological sample is blood, lymphocyte, monocyte, neutrophil, basophil, eosinophil, myeloid lineage cell, lymphoid lineage cell, bone marrow, saliva, buccal swab, nasal swab, urine, fecal material, hair, breast tissue, ovarian tissue, uterine tissue, cervical tissue, prostate tissue, testicular tissue, brain tissue, neuronal cell, astrocyte, liver tissue, kidney, thyroid tissue, stomach tissue, intestine tissue, pancreatic tissue, vascular tissue, skin, lung tissue, bone tissue, cartilage, ligament, tendon, fat cells, muscle cells, neurons, astrocytes, cultured cells with different passage number, cancer/tumor cells, cancer/tumor tissue, normal cells, normal tissue, any tissue(s) or cell(s) with a nucleus containing genetic material.

Item 40: Method of screening for potential therapeutic activity of the molecule or compound or pharmaceutical composition or other intervention (e.g., but not limited to diet, physical activity, food, food supplement, medical device, device etc.) against aging related condition or disease, comprising method of any one of preceding items.

Item 41: Method of screening for potential therapeutic activity of the molecule or compound or pharmaceutical composition or other intervention (e.g., but not limited to diet, physical activity, food, food supplement, medical device, etc.) against aging related condition or disease, comprising method of any one of preceding items, wherein the biological age is measured before and after the intervention, optionally after passing of 10% of average life span of the mammal of the same phenotype. In some embodiments, the intervention is considered as having effect against aging, aging related condition or disease if biological age of mammal administered such intervention in therapeutically effective dosage is less than in a control group of mammals.

Item 42: Method of screening of toxicity of the molecule or compound or pharmaceutical composition or other intervention (e.g., but not limited to diet, physical activity, food, food supplement, medical device, device, life style, etc.), comprising method of any one of preceding items.

Item 43: Method of screening of toxicity of the molecule or compound or pharmaceutical composition or other intervention (e.g., but not limited to diet, physical activity, food, food supplement, medical device, device, life style etc.), comprising method of any one of preceding items, wherein the biological age is measured before and after the intervention, optionally after passing of 10% of average life span of the mammal of the same phenotype. In some embodiments, the intervention is considered as having toxic or adverse effect, if biological age of mammal administered such intervention in therapeutically effective dosage is bigger than in a control group of mammals.

Item 44: Method of any one of preceding items, wherein such method further comprises the determination of derived parameter from the biological age. In some embodiments such derived parameter is selected from the group consisting of a frailty index, a physiological resilience, a survival function, a force of mortality, a life expectancy, a life expectancy from birth, and a remaining life expectancy of the mammal.

Item 45: Method of any one of preceding items, wherein such method is implemented in computer.

Item 46: A tangible medium, configured with instructions that when executed cause a processor to perform the method of any one of preceding items.

Item 47: A tangible medium, configured with instructions that when executed cause a processor to perform the method of any one of preceding items, wherein such tangible medium comprises a non-transitory computer readable medium.

Item 48: The apparatus, tangible medium, computer chip or method any of preceding items, wherein a determination of biological age, is performed in response to the received plurality of values of health parameters.

Item 49: A computer system comprising a processor; and a memory configured with instructions that cause said processor to apply a method of any one of preceding items.

Item 50: A computer system for implementation of method of any one of preceding items, the computer system comprising a processor; and a memory configured with instructions that cause said processor to apply a corresponding method.

Item 51: A computer system for biological age determination, the computer system comprising a processor; and a memory configured with instructions that cause said processor to apply a corresponding method.

Item 52: A computer system for toxicity prediction, the computer system comprising a processor; and a memory configured with instructions that cause said processor to apply a corresponding method.

Item 53: A computer software product, said product configured for determination of biological age or predicting drug efficacy for treating a disorder in a patient or predicting toxicity of the intervention, the product comprising a computer-readable medium in which program instructions are stored, which instructions, when read by a computer, cause the computer to:
a. receive values of health parameters,
b. determine biological age from received values of health parameters by at least one of
the methods of any one of the preceding items;
c. output the value of determined biological age.

Item 54: A computer software product implementing method of any of preceding items.

Item 55: A computer software product, which instructions, when read by a computer, cause the computer to implement method of any of preceding items.

Item 56: The apparatus, tangible medium, computer chip or method any of preceding items, wherein a determination of biological age, is performed in response to the received plurality of values of health parameters and based on instructions and parameters generated using machine learning techniques for determining the biological age for the mammal.

Item 57: A system comprising:
a module configured to receive values of health parameters;
a storage assembly configured to store input and output information from the determination module;
a module adapted to determine biological age from the values of health parameters, and to provide a output of value of biological age; and an output module for displaying the information related to biological age for the user.

Item 58: Any one of preceding items, wherein algorithm is built with the use of Neural network.

Item 59: Any one of preceding items, wherein algorithm is built with the use of Neural network which is implemented using python 3 and tensorflow framework.

Item 60: Any one of preceding items, wherein instead of biological age a hazard ratio is determined.

Some other non-limiting examples of this invention and further disclosure of the invention are provided in the section titled “Identification of a Blood Test-Based Biomarker of Aging through Deep Learning of Aging Trajectories in Large Phenotypic Datasets of Mice.”

### Identification of a Blood Test-Based Biomarker of Aging Through Deep Learning of Aging Trajectories in Large Phenotypic Datasets of Mice

We proposed and characterized a novel biomarker of aging and frailty in mice trained from the large set of the most conventional, easily measured blood parameters such as Complete Blood Counts (CBC) from the open-access Mouse Phenome Database (MPD). Instead of postulating the existence of an aging clock associated with any particular subsystem of an aging organism, we assumed that aging arises cooperatively from positive feedback loops spanning across physiological compartments and leading to an organism-level instability of the underlying regulatory network. To analyze the data, we employed a deep artificial neural network including auto-encoder (AE) and auto-regression (AR) components. The AE was used for dimensionality reduction and denoising the data. The AR was used to describe the dynamics of an individual mouse's health state by means of stochastic evolution of a single organism state variable, the “dynamic frailty index” (dFI), that is the linear combination of the latent AE features and has the meaning of the total number of regulatory abnormalities developed up to the point of the measurement or, more formally, the order parameter associated with the instability. We used neither the chronological age nor the remaining lifespan of the animals while training the model. Nevertheless, dFI fully described aging on the organism level, that is it increased exponentially with age and predicted remaining lifespan. Notably, dFI correlated strongly with multiple hallmarks of aging such as physiological frailty index, indications of physical decline, molecular markers of inflammation and accumulation of senescent cells. The dynamic nature of dFI was demonstrated in mice subjected to aging acceleration by placement on a high-fat diet and aging deceleration by treatment with rapamycin.

An ever-increasing number of physiological state variables, such as blood cell counts and blood chemistry [1-4], DNA methylation [4-8], locomotor activity [4, 9-11], and exploratory behavior [9, 12], have been investigated in association with aging and used to quantify aging progression in in-vivo experiments and in future anti-aging clinical trials [4]. Most of the common statistical models used in aging studies require chronological age or age at death as labels for training; however, this data are rarely available in sufficient quantity from human and laboratory animal cohorts. Even less information is available regarding the change in biomarkers of aging and frailty across the lifespan of individual animals or patients in response to lifespan-modifying interventions. Thus, the field would benefit from development of a convincingly justified easily measurable and reliable biomarker of aging ideally obtainable from conventional and automated measurements such as routine blood tests.

To produce a quantitative description of the aging process in mice, we turned to the largest open-access source of phenotypic data, the Mouse Phenome Database (MPD) [13, 14]. The MPD contains a wide range of phenotype data sets including behavioral, morphological and physiological characteristics and involving a diverse set of in-bred mouse strains. In the present work, we implemented biomarker of aging based on complete blood cell (CBC) measurements. CBC test is a well established, easily obtainable laboratory analysis protocol and it has a long list of applications in both clinical medicine and biomedical research [15].

Principal Components Analysis (PCA) revealed that the fluctuations in CBC variables in the MPD are dominated by the dynamics of a single cluster of features, jointly deviating from the initial state and increasing in variance with age.

The behavior is typical for non-equilibrium complex systems with strong interactions between the components operating close to the critical or tipping point separating the stable and the unstable regimes [16]. Under the circumstances, the organism state fluctuations should be driven by the dynamics of a single variable, that is the organism-level property having the meaning of the or-der parameter corresponding to the unstable phase [17] and associated with aging drift and mortality acceleration [18].

To generate the biomarker of aging, we built a state-of-the-art deep artificial neural network composed of de-noising autoencoder (AE) and the auto-regressive (AR) model, that is a computational metaphor for the dynamics of the order parameter. The network output variable exhibited the most desirable properties of a biological age marker: it increased exponentially with age, predicted the remaining lifespan of the animals, correlated with multiple hallmarks of aging, and was henceforth referred to as the dynamic frailty index (dFI). The dynamic char-acter of dFI was demonstrated in experiments involving treatments previously shown to accelerate (high-fat diet) or decelerate (rapamycin) aging in mice.

Therefore, we conclude that dFI is an accurate, easily accessible biological age proxy for experimental characterization of aging process and anti-aging interventions. On a more conceptual level, our work demonstrates that the auto-regressive analysis provided by the AE-AR deep learning architecture may be a useful tool for the fully un-supervised (label-free) discovery of biological age markers from any type of phenotypic data involving longitudinal measurements.

Results

A. Overview of Aging in the Mouse Phenome Database

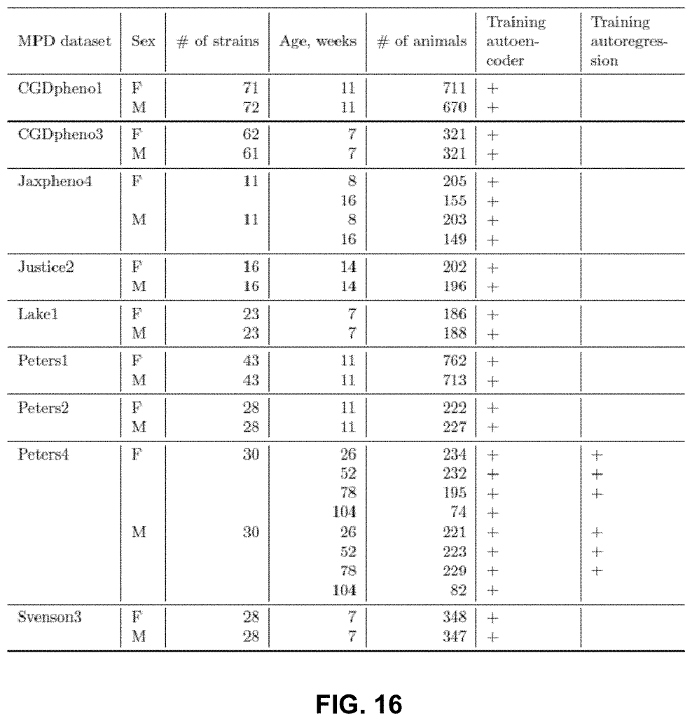

We started by building a training set from the largest publicly available source of phenotypic data, the Mouse Phenome Database (MPD) [13, 14]. To achieve the best possible compatibility with earlier studies, we scanned the database records to maximize the number of available measurements common to those used in the construction of the physiological frailty index (PFI) in [19]. As a result, we chose a subset of twelve complete blood count (CBC) measurements from nine datasets, altogether including more than 7, 500 animals (see FIG. 16 for a complete list of datasets used for training of the models disclosed herein).

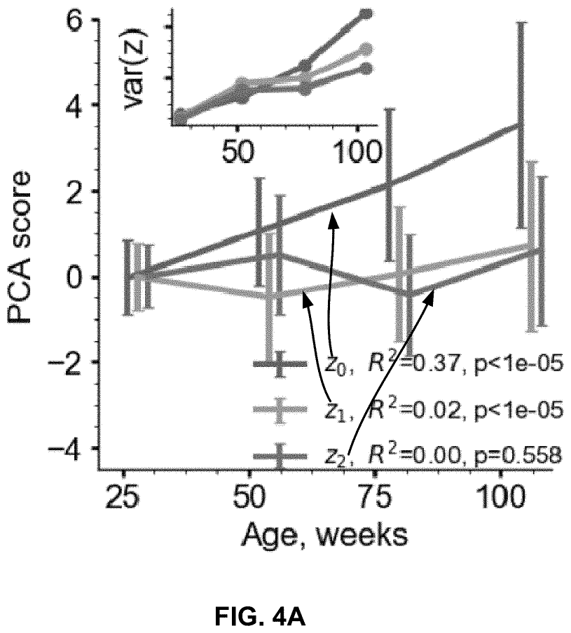

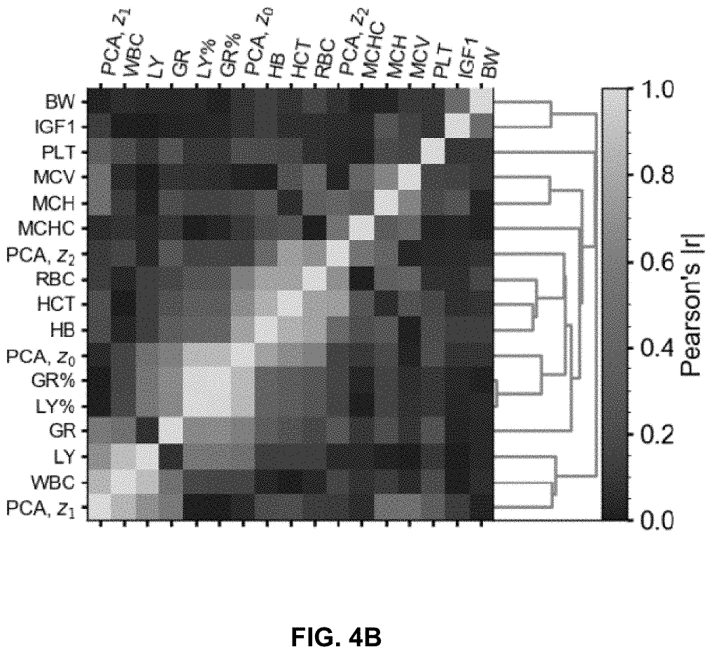

To visualize the 12-dimensional CBC data from the MPD, we performed principal component analysis (PCA), that is a computational technique commonly used for multivariate data analysis [20-22]. PCA of the MPD slice representing fully-grown animals (exceeding the age of 25 weeks old) turned out to be particularly simple, see FIG. 4A. In this case, most of the variance in the data (27%) is explained by the first principal component (PC) score, z0, which is strongly associated with age. None of the subsequent PC scores (z1, z2, etc., each explaining 20%, 16%, etc. of the data variance, respectively) showed any rea-sonable correlation with age. However, each of the PC scores was associated with a distinct cluster of biologically related blood features, as shown in FIG. 4B. For example, the first two PC scores could be predominantly connected with the red and white blood cell counts, respectively.

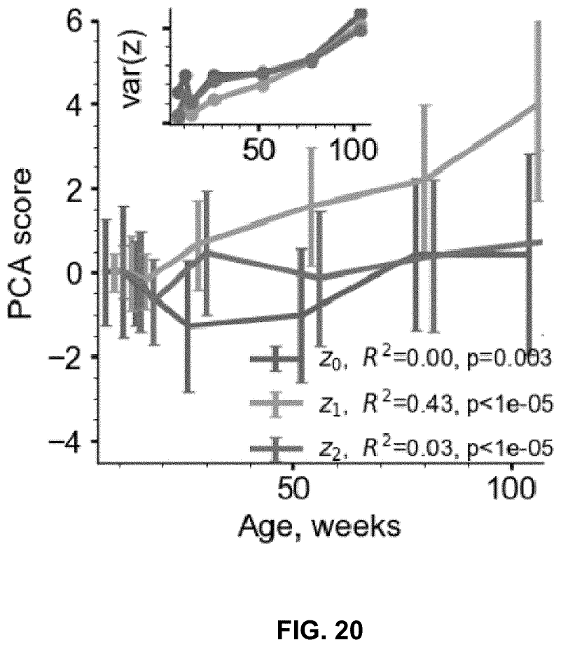

Notably, the largest variance in the data representing the full dataset was rather associated with animal growth and maturation. The first PC score is associated with the age of animals younger than 25 weeks old but does not change substantially after that age, see FIG. 20. The second, PC score exhibited an association with age over the entire available age range. This means that aging and early development in mice are different phenotypes and henceforth we perform all our calculations using the data from animals aged older than 25 weeks.

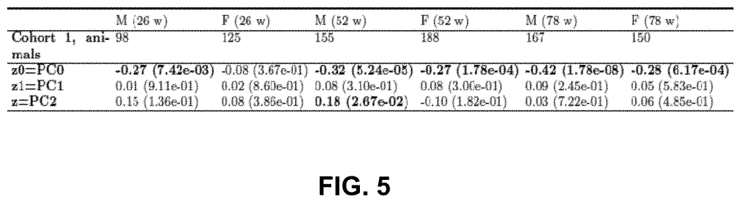

The first PC score, z₀, was the only PCA variable as-sociated with the remaining lifespan of the animals. This was determined by using Spearman's rank-order correlation tests to evaluate potential associations between the first three PC scores and the age at death within cohorts of mice of the same age and sex (see FIG. 5). The z₀ variable had therefore the most desirable properties of biological age.

Variance of the PCA scores and hence the biological age also grew with age (see the inset in FIG. 4A), which is a signature of stochastic broadening. The dimensionality reduction revealed by the PCA and the association of the large-scale fluctuations driving the slow evolution or disintegration of the system are characteristic of criticality, which is a special case of the dynamics of a complex system unfolding near a bifurcation or a tipping point, on the boundary of a dynamic stability region [16, 23]

B. Aging, Critical Dynamics of the Organism's State and the Dynamic Frailty Index (dFI)

The dynamics of the order parameter associated with the unstable phase is a measure of the aging drift and mortality acceleration in aging organisms [18] and henceforth is to be referred to as the dynamic frailty index (dFI). In this section, we summarize the necessary theoretical framework required for identification of the biomarker and quantitative description of aging in biological data.

Over sufficiently long time-scales, the fluctuations of physiological indices (such as CBC features), xᵢ, are expected to follow the dynamics of the order parameter, z=dFI: xᵢ=bᵢz+ξᵢ. Here ξᵢ is noise, bᵢ is a vector that may differ across species, and the integer index i enumerates the measured features.

Close to the tipping point, the dynamics of the physio-logical state is slow and hence the variable z satisfies the stochastic Langevin equation with the higher order time derivative terms neglected:

z′=αz+gz²+f. (Equation 1)

Here the linear term, αz, on the right side of the equation represents the effect of the regulatory network stiffness governing the responses of the organism to small stresses producing small deviations of the organism state from its most stable position. The following term, gz², represents the lowest order non-linear coupling effects of regulatory interactions.

The stochastic forces f represent external stresses and the effects of endogenous factors not described by the effective Equation 1. Naturally, we assume that random perturbations of the organism state are serially uncorrelated, so that (f (t)f(t^{t})˜B, where B is the power of the noise, and ( . . . ) stands for averaging along the aging trajectory.

The equation establishes the “law of motion” for the organism's physiological state It is a mathematical relationship between the rate of change of the organism state variable, z′=dz/dt, on the left side of the equation, and the effects of deterministic (αz, gz²) and stochastic forces (f), on the right side.

Depending on the sign of the stiffness coefficient, α, the organism state may be dynamically stable (if α<0) or unstable (if α>0). In the latter case, small deviations of the organism state get amplified over time so that no equilibrium is possible and the solution of Eq. 1 describes an aging organism. Typically, a is small, and hence, the evolution of the physiological indices exhibits hallmarks of critically: it is slow (critical slowing down) and the fluctuations of the physiological state following the variations in z are large [z²˜B exp(2αt)/2α (critical fluctuations).

Very early in life, the deviations from the critical point are small and the evolution of the organism state is dominated by diffusion. Later in life, the linear term takes over such that the deviations from the youthful state accelerate exponentially:

z≈z⁻exp(αt)+z₀. (Equation 2)

where z⁻˜(B/α)^{1/2} and z₀ are constants representing the accumulated early effects of random and deterministic forces, respectively.

Finally, once dFI is sufficiently large, z>Z=α/g, the non-linear terms take over, disintegration of the organism state proceeds at a rate greater than exponential, and the animal dies in a finite time. Mortality in this model increases up to the average lifespan t⁻=1/α log(Z/z⁻). Mortality is a complex function of the order parameter z and hence of the chronological age. The mortality acceleration rate at the age corresponding to the average lifespan is of the same order of a.

C. Identification of dFI from Longitudinal Data by Applying a Deep Neural Network

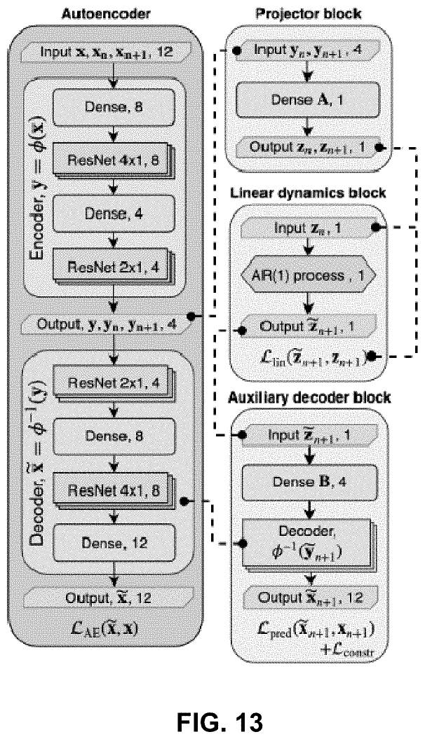

To identify the dFI from CBC measurements we performed a fit of the experimental data from MPD onto solutions of Eq. 1 with the help of an artificial neuron network. Altogether we used 7616 samples from 9 MPD datasets as the training set (see Material and Methods and FIG. 16). We employed a combination of a deep auto-encoder (AE) and a simple auto-regression (AR) model for modal analysis (AE-AR; see neural network architecture in FIG. 13). At its bottleneck, the encoder arm of the AE produced a compressed 4-dimensional representation y of the input, the 12-dimensional physiological state vectors x built from the available CBC measurements. The decoder arm reconstructed the original 12-dimensional state x⁻ from the bottle-neck features.

The longitudinal slice of MPD has only a few hundred of specimen with serial measurements. The combined AE-AR approach adopted here let us maximize the number of mice used for training of the complete model. Thus, we were able to use all the available samples, including both the cross-sectional and the longitudinal segments of the MPD, in the AE arm of the algorithm to produce the highest quality low-dimensional representation of the data.

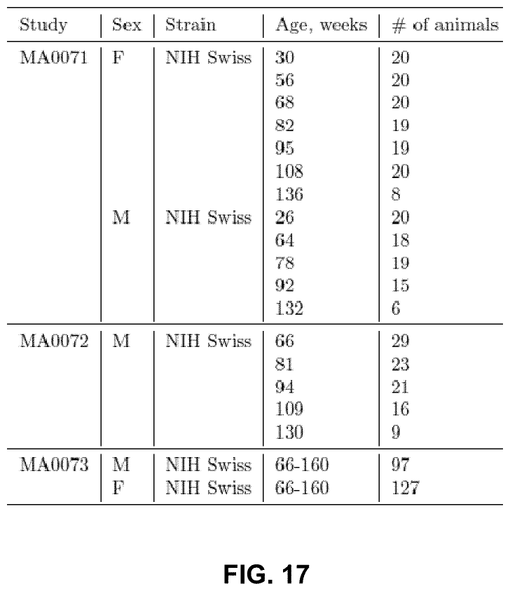

The performance of the models was validated in test datasets (see Material and Methods and FIG. 17), which were completely excluded from fitting. The test datasets were obtained from independent experiments by collecting CBC samples from cohorts of NIH Swiss mice of different age and sex (dataset MA0071), cohort NIH Swiss male mice observed for 15 months (dataset MA0072) and cohorts of naive male and female NIH Swiss mice that were humanely euthanized after reaching approved experimental endpoints (dataset MA0073).

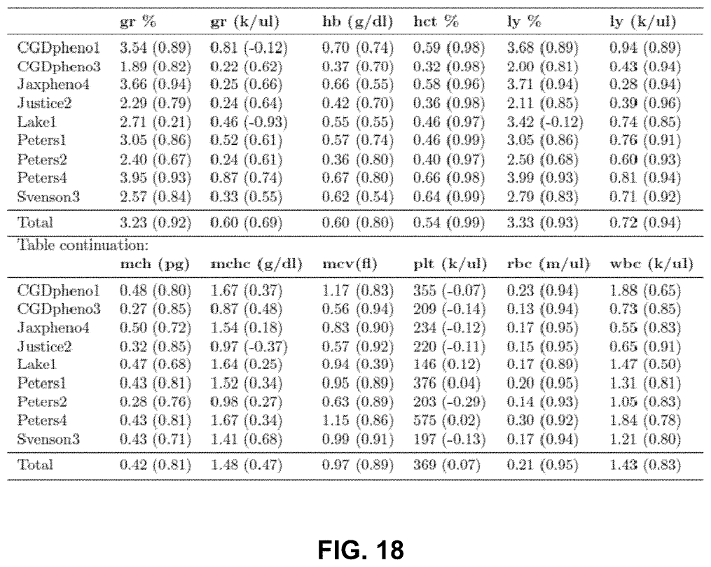

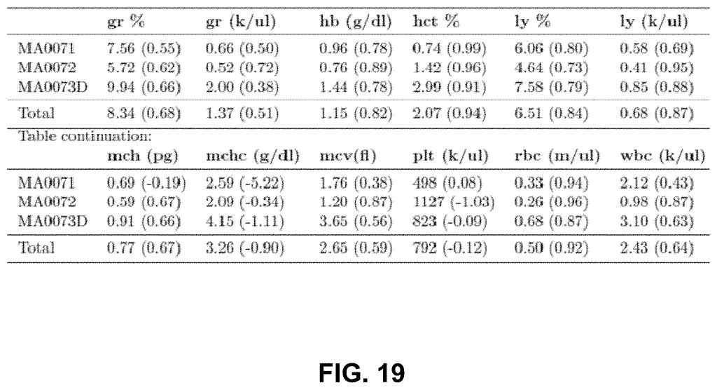

We estimated the reconstruction error of the AE by calculation of the root mean squared error (RMSE) and coefficient of determination R2 for each CBC feature in training and test sets (see FIG. 18 and FIG. 19). The average RMSE in the test set was 228.8 with R2=0.54; in the training set, RMSE was 106.4 and R2=0.77. The best reconstruction was achieved for hematocrit (R2=0.94), red blood cells (R2=0.92) and lymphocytes (R2=0.87); the worst results were for mean corpuscular hemoglobin concentration (R2=−0.9) and platelets (R2=−0.12) in the test set.

Simultaneously with the AE, we trained the network to fit the longitudinal slice of MPD (including fully-grown animals at ages from 26 to 104 weeks with a sampling interval of Δt=26 weeks) to the solution of the linearized (g=0) version of Equation 1:

z(t+Δt)=rz(t)+z′+ξ (Equation 3)

where z is the best possible linear combination of AE bottle-neck features. The state z is the output of the algorithm, the estimation of dFI (refer to the detailed description of the artificial neural network architecture behind the AE-AR algorithm in FIG. 13). The constants r=exp(αΔt)≈1, zt, and ξ which are the best fit values of the autoregression coefficient, the constant shift, and the error of the fit (the combination of the system's noise and measurement errors), respectively.

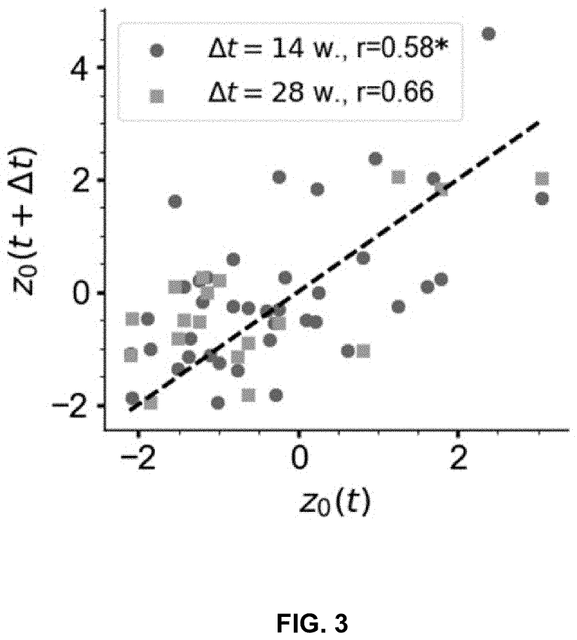

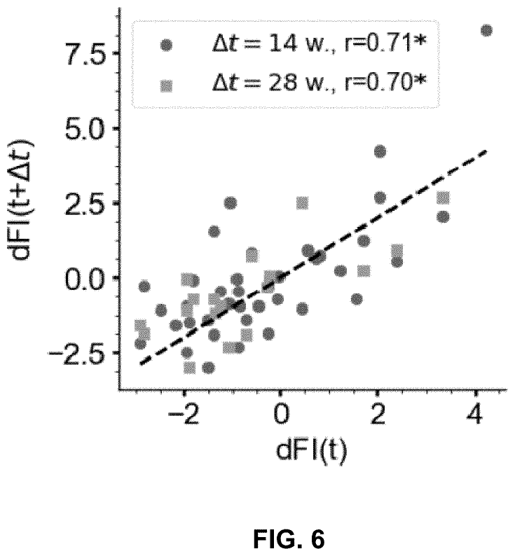

Performance of the AR model was demonstrated by plotting the autocorrelations between dFI values measured along aging trajectories of the same mice at age points separated by 14 and 28 weeks in the test dataset MA0072 (see FIG. 6). Remarkably, the correlations (Pear-son's r=0.71 (p<0.001) and r=0.70 (p<0.001)) of the age-adjusted dFI persisted over the time lags of 14 and 28 weeks. The dFI auto-correlations were better than the autocorrelations of the first PC score z0 for the same mice, see FIG. 3; the corresponding Pearson's correlation values were r=0.58 (p<0.001) and r=0.66 (p=0.002) for 14- and 28-week time lags, correspondingly.

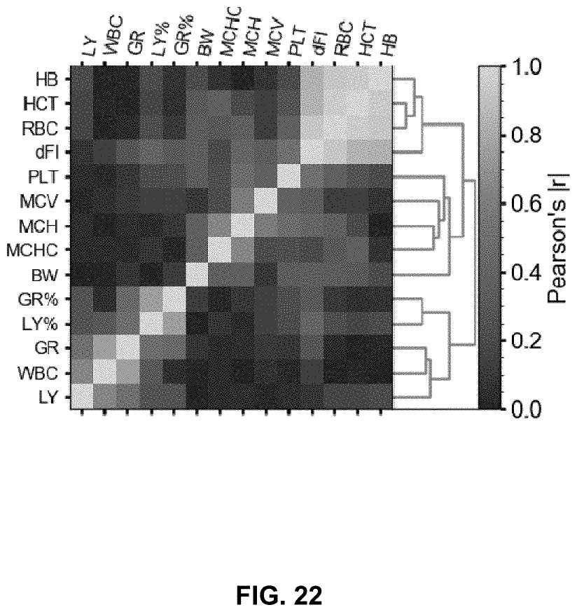

A semi-quantitative view of hierarchical clustering of CBC features co-variances in the test dataset produced groups of features associated with the immune system (white blood cell counts and the related quantities), metabolic rate/oxygen consumption (red blood cell counts and hemoglobin concentrations), and an apparently independent subsystem formed by platelets (see FIG. 22).

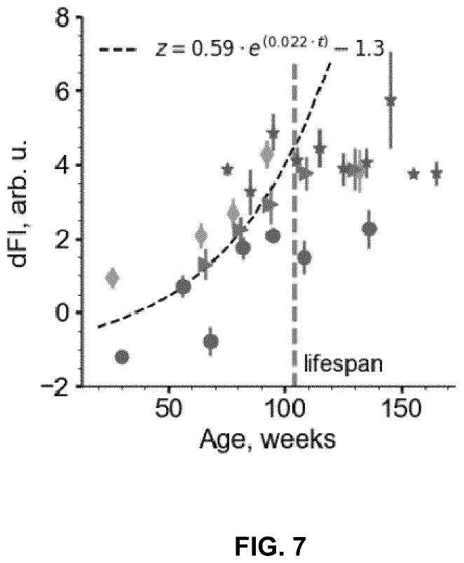

dFI was associated with animal age in both the training and test (see FIGS. 17 and 7, respectively). As expected from the qualitative solution of Equation 1, dFI increased up to the age corresponding to the average animal lifespan (approximately 100 weeks in our case). We performed an exponential fit in the form of Equation 2 on the data from the test datasets (excluding animals that lived longer than the strains average lifespan and animals at the end of their life from the dataset MA0073). The calculation returned dFI growth exponent of α=0.022 per week. This estimate is somewhat smaller than (but still of the same order as) the expected Gompertz mortality acceleration rate of 0.037 per week [24] for the SWR/J strain.

Saturation of the dFI beyond the average lifespan in the training and test datasets revealed a limiting value that is apparently incompatible with the animals' survival. This possibility can be highlighted by plotting the dFI ranges from a separate cohort of “unhealthy” mice from MA0073 experiment, representing the animals scheduled for euthanasia under lab requirements (FIG. 7, stars).

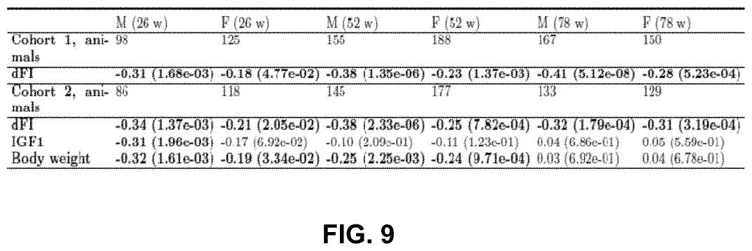

The long autocorrelation time of dFI together with its exponential growth at a rate compatible with the mortality acceleration rate are indicators of the association between dFI and mortality. This was further supported by the Spearman's rank correlation between the dFI values and the order of the death events within mice in cohorts of same age and sex (see FIG. 9). We obtained significant correlations for dFI and remaining lifespan for all cohorts. Importantly, the age- and sex-adjusted dFI predicted remaining lifespan better than a naive PC score z0 from the linear analysis.

The dFI predicted remaining lifespan later in life better than body weight or insulin-like growth factor 1 (IGF1) serum level, which were previously shown to be associated with mortality in [25] and [26]. As pointed out in [26] and checked here, the concentration of IGF1 in serum was significantly associated with lifespan (r=−0.28, p=0.008) only in one cohort of younger, 26-week old male mice. According to [25] and our calculations, mouse body weight is better associated with mortality, again, in the youngest animals at the ages of 26 and 52 weeks.

D. dFI and Hallmarks of Aging

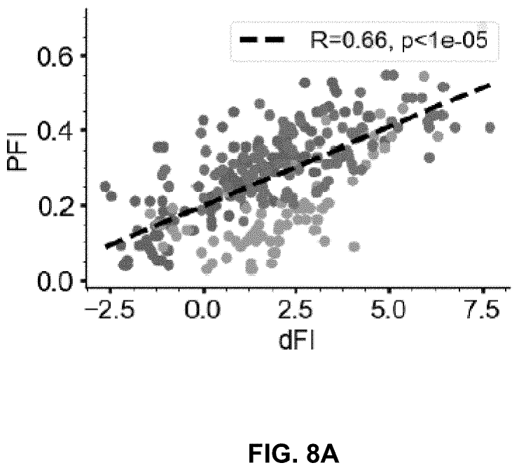

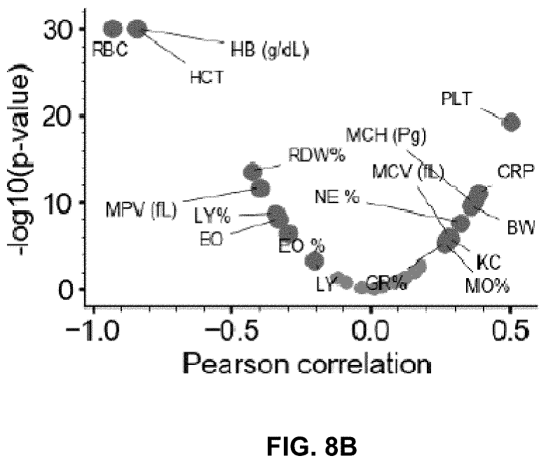

To further validate dFI as an age biomarker, we examined its association with physiological frailty index (PFI), a quantitative measure of aging and frailty established previously [19]. dFI and PFI were found to be strongly correlated (Pearson's r=0.64, p<0.001), see FIG. 8A. PFI is a composite frailty score and depends on CBC measures for its determination. PFI is also influenced by changes in more traditional measures of frailty, such as grip strength, cardiovascular health, inflammation markers, etc. Remarkably, the correlation between PFI and dFI remained significant after adjustment for sex and age (Pearson's r=0.54, p<0.001).

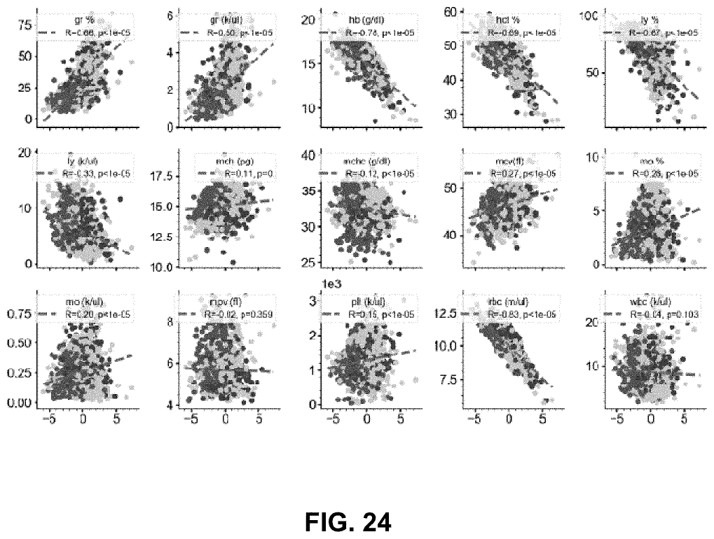

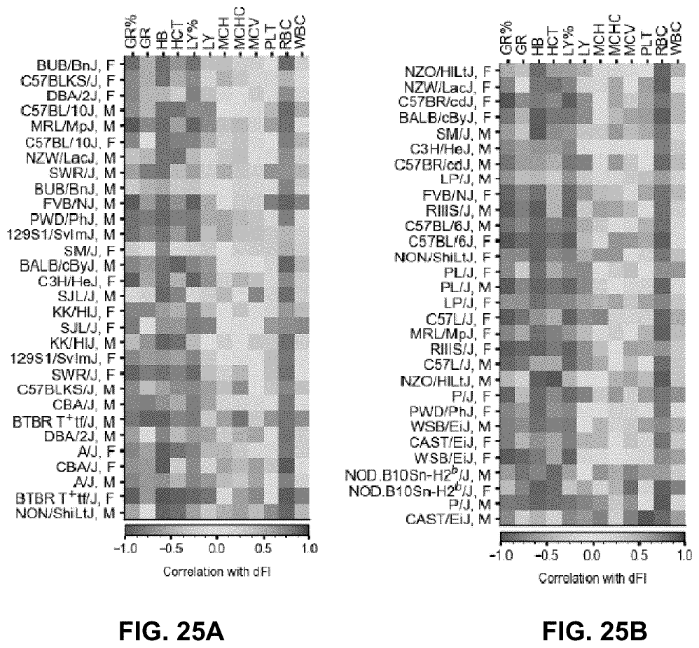

As illustrated in FIGS. 22 and 24, we observed that the dFI was significantly associated with an extended set of CBC features across independent functional subsystems (most notably, but not limited to, myeloid cell lineage). The correlation between dFI and myeloid cell features was less profound in the training set, involving multiple strains (see FIG. 24). The correlation coefficient is a measure of response of dFI to individual CBC features variation and is different (sometimes even of opposite sign) in various mouse strains, see FIGS. 25A and 25B. The variation of the associations of individual features and dFI would be a significant challenge to a linear model and is a demonstration of the non-linear character of the autoencoder.

The dFI was strongly associated with red blood cell distribution width (RDW) and body weight (FIG. 8B), known predictors of frailty in both mice [15] and humans [27, 28]. dFI was also strongly associated with levels of C-reactive protein (CRP, r=0.39, p<0.001) and the murine chemokine CXCL1 (KC, r=0.28, p<0.001), both of which are known markers of systemic inflammation and mortality [29-31].

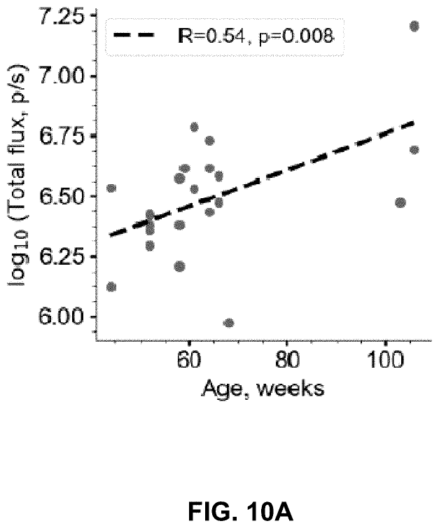

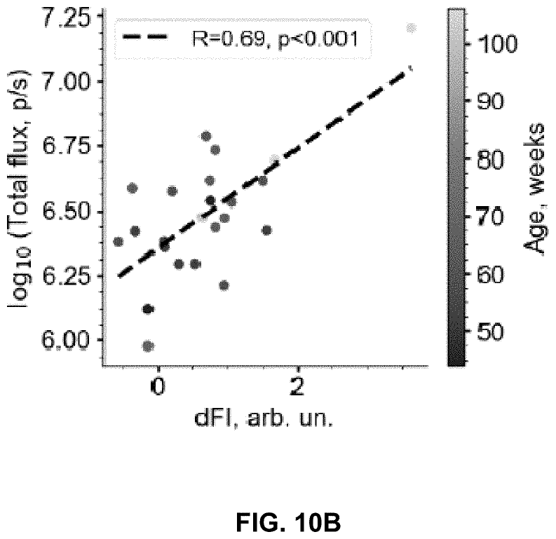

Aging is associated with an increasing burden of senescent cells [32, 33], widely considered to be a source of chronic sterile systemic inflammation, “inflammaging” [34]. Senescent cells are commonly detected in vivo as a population of p16/Ink4a-positive cells accumulated with age recognized by the activity of p16/Ink4a promoter-driven reporters [35]. We utilized earlier described hemizygous p16/Ink4a reporter mice with one p16/Ink4a allele knocked in with firefly luciferase cDNA [36]. FIG. 10A shows the correlation between animal age and presence of senescent cells, as measured by the flux from p16/Ink4a promoter-driven luciferase activity (r=0.54, p=0.01). The correlation of this SC proxy (total luciferase flux) with dFI was even stronger (r=0.69, p<0.001; see FIG. 10B).

E. dFI Reflects Lifespan-Modulating Interventions

Having established the association between dFI and remaining lifespan in the MPD, we next tested its predictive power by evaluating the response of dFI to life-long interventions known to affect the lifespan of mice. In the data from [19], male mice that were fed a high-fat diet (HFD) instead of a regular diet (RD) beginning at 50 weeks of age had significantly reduced lifespans (FIG. 11A) and also showed a significant increase in average dFI measured at week 78 (p=0.05, Student's two-tailed t-test; FIG. 11B) in comparison to control RD-fed males. In contrast, HFD feeding of female mice had no effect on either lifespan or average dFI (FIGS. 11C and 11D). Thus, dFI appeared to be a good predictor of gender-dependent differences in organismal aging response to HFD, the underlying reasons for which remain to be explained.

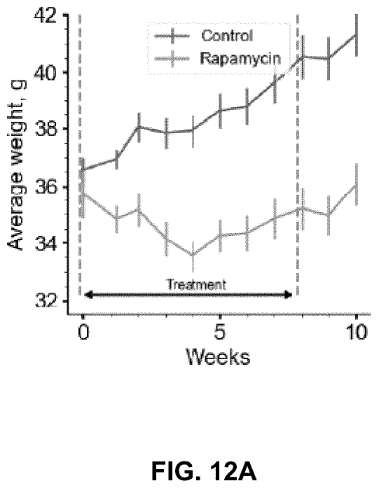

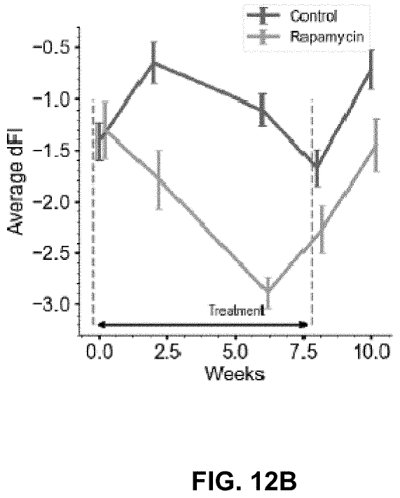

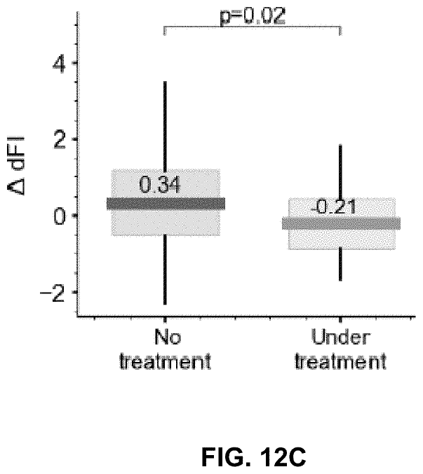

We also tested the response of dFI to a short lifespan-extending condition: treatment with rapamycin [37, 38]. Here we present the results of an experiment with 60-week-old male mice treated with rapamycin daily at a dose of 12 mg/kg for 8 weeks or, in the control group, vehicle on the same schedule. The cohort of 24 60-week old C57BL/6 male mice was divided into treatment and control groups using a stratified randomization technique to produce indistinguishable distributions of dFI values. Body weights were measured every week and increased as expected in the control group (see FIG. 12A). In contrast, body weight in the rapamycin-treated group stayed ap-proximately constant near the initial value throughout the observation period of 10 weeks. A lower body weight is typical for rapamycin-treated mice in comparison to control group [19, 39]. In order to generate dFI values for the mice in this experiment, blood samples were collected from each animal for CBC measurements every two weeks (see FIG. 12B).

The longitudinal character of sampling in the experiment let us use the autoregression analysis to detect the effects of the drug on the dynamics of dFI in the course of the experiment. Whenever a non-random force (that is the effect of the drug) is present in Equation 1, the jump in dFI between any of consequent measurements from the same animal should satisfy modified Equation 3:

z(t+Δt)=rz(t)+z^{t}+J+ξ (Equation 4)

where J is the accumulated effect of the drug along the aging trajectory. The time intervals between the sub-sequent measurements are very small, αΔt<<1 and hence the autoregression coefficient r≈1. We therefore expected to identify the effect of rapamycin by comparing the distributions of the dFI increments between the measurements.

We indeed observed the dFI jumps that were significantly different depending on whether rapamycin treatment was present between the dFI measurements both in the control and the treated groups, see FIG. 12C (p=0.02, Student's two-tailed test). These results support the possibility of using longitudinal dFI measurements to detect effects of life-extending therapeutics over much shorter times that what could be done based on the appearance of evident changes in frailty or longevity.

We introduced a novel way of using deep artificial neuronal networks to train biomarkers of age and frailty from big biomedical data involving longitudinal measurements, i.e., multiple samples of the same animals collected along the aging trajectories. We exemplified the approach with the discovery and characterization of a novel biomarker of aging in mice, the dynamic frailty index (dFI), from conventional and automated measurements of Complete Blood Counts (CBC) and trained from the data from Mouse Phenome Database (MPD).

We started with linear dimensionality reduction using the principal component analysis (PCA), which has a long list of applications in biomarkers of aging research [40, 41]. As expected, we observed that the variance of CBC features in MPD is dominated by a cluster of features closely associated with the first PC score; none of the other PC scores correlated with age. Hence, the data suggests that aging in mice can be explained by the dynamics of a single (latent) variable that is a single organism-level quantity and a natural indicator of the progress of aging (i.e., a biomarker of aging).

The associations of slow organism state dynamics with the first principle component score is a hallmark of criticality, that is the situation whenever a system's dynamics occurs in the vicinity of a tipping (or critical) point, separating the stable and the unstable regime [42]. Gene regulatory networks of most species are tuned to criticality [42]. In [18] we proposed that aging corresponds to the unstable regime, when small deviations of the organism state from its initial position get amplified exponentially. The first principal component score is then an approximation to the order parameter, herein referred to as dFI, that is corresponding to the unstable phase and having the meaning of the total number of the regulatory errors accumulated in the course of life of the animal [43].

The order-parameter is a generalization of a concept originally introduced in the Ginzburg-Landau theory in order to describe phase-transitions in thermodynamics [44]. The order parameter concept was further generalized by Haken to the “enslaving-principle” saying that next to the critical point the dynamics of fast-relaxing (stable) components of a system is completely determined by the ‘slow’ dynamics of only a few ‘order-parameters’ (often variables associated with unstable modes) [17]. The dFI identified in connection with the dynamics of the order parameter is then not a mere ma-chine learning tool for specific predictions, but a fundamental macroscopic property of the aging organism as a non-equilibrium system.

PCA belongs to the class of unsupervised learning algorithms, such that the model does not require any la-bels such as chronological age or the remaining lifespan for its training. It is therefore remarkable, although expected from a large corpus of previous works, that the first principle components are associated with age and the remaining lifespan of the animals. However, the abilities of linear rank reduction techniques, such as PCA, to recover accurate dynamic description of aging is limited for the following reasons. First, there are no reasons to believe that the effects of non-linear interactions be-tween different dynamic subsystems are small. That is why the result of such a procedure cannot be expected to perform well in different biological contexts (strains, laboratory conditions, or therapeutic interventions such as drugs).

Second, biological measurements are often noisy, and hence, simple techniques lacking efficient regularization may fail to reconstruct the latent variables space correctly unless a prohibitively large number of samples is obtained [45]. Finally, the association of the first principal component with the order parameter and hence the biomarker of aging in the form of dFI is only an ap-proximate statement. Fundamentally, there is no way to identify the dynamics of the system from the data, that does not include the dynamics itself in the form of multiple measurements of the same organism along the aging trajectory.

To compensate for the drawbacks of PCA, we employed an artificial neuron network, a combination of a deep de-noising auto-encoder (AE) and an auto-regressive (AR) model. The AE part of the algorithm is a non-linear generalization of PCA and was used to compress the correlated and necessarily noisy biological measurements into a compact set of latent variables, a low-dimensional representation of the organism state.

The AR-arm of the network is nothing else but the best possible prediction of a future state of the same animal from the current measurements in such a way that the collective variable inferred by the model is a directly interpretable and physiologically relevant feature, the dynamic frailty index (dFI). The approach is a computational metaphor for the analytical model behind identification of the order parameters associated with the organism-level regulatory network instability from [18].

The neural network applied here was inspired by deep rank-reduction architectures, recently used for characterization and interpretation of numerical solutions of large non-linear dynamical systems [46, 47].

dFI increases exponentially with age and is associated with remaining lifespan. It is therefore a natural quantitative measure of aging drift and hence may be used as a biomarker of age. Remarkably, it appears that blood parameter data alone can define biological age with a degree of accuracy comparable to that of the best previously described biomarkers of aging e.g., DNA methylation-based clock [5-8] or physiological frailty index [19]. This may reflect a key role for aging of hematopoietic tissue in determining aging of the whole organism, a concept that is intuitively acceptable given the universal systemic physiological function of blood.

As an alternative explanation, age-dependent changes in blood parameters may be secondary events induced by aging of the remainder of the organism (i.e., various solid tissues). However, accumulated experimental evidence argues against this. In fact, there are multiple reports demonstrating rejuvenating effects of young hematopoietic system on old animals delivered either by bone marrow transplantation or by parabiosis (reviewed in ref. [48]). Moreover, restoration of mouse hematopoiesis through transplantation of HSCs from young vs old donors clearly demonstrated that aged HSCs cannot be rejuvenated by the environment of a young body [49]. Also, the interpretation of age dependence of HSC-derived features as secondary effects of aging would face formal difficulties, since the dynamics of such factors should exhibit shorter, in fact at least twice shorter, doubling times than the dFI and the mortality rate doubling times.

A peculiar result of our analysis is that our data strongly point towards myeloid lineage that provides much more accurate predictors of biological age than lymphoid lineage parameters. This is counterintuitive since aging is generally accepted to be associated with the well-documented general decline in immunity known as an immunosenescence [50-52], the phenomenon illustrated by the reduced efficiency of vaccination [53] and increased frequency and lethality of infectious diseases and cancer in older organisms [54]. Nevertheless, there is strong experimental evidence that supports and provides a mechanistic explanation for our finding that myeloid parameters weigh more heavily than lymphoid ones as biological age indicators. In a comprehensive study of the epigenetic mechanisms of HSC aging, Beerman et al. [49]. described age-dependent epigenetic reprogramming that leads to a significant shift towards myeloid lineage differentiation of the progeny of aged HSCs [49, 55, 56]. This shift is driven by specific changes in methylation of the DNA of HSCs that occur during mouse aging. Surprisingly, these changes in methylation, which alter gene expression, do not occur in the part of the genome that controls HSC phenotype, but rather modify DNA regions encoding genes that control downstream differentiation stages. Remarkably, the pattern of DNA methylation changes associated with aging of HSCs seems to represent the same process that was previously described as a DNA methylation-based clock [5, 49], and therefore, may be part of the same epigenetically controlled fundamental aging mechanism. Another factor that could diminish the impact of lymphoid lineage-related parameters as biological age markers is the reactive nature of this branch of hematopoiesis, which serves to rapidly respond to sporadic events such as viral or bacterial infection, wounding, and other types of stress requiring an emergency response usually in the form of acute inflammation. Since the time of occurrence of such events is unpredictable, age-associated changes may be masked by the noise coming from large-scale age-unrelated fluctuations in the lymphoid compartment.

These observations do not mean that the blood is the single determinant of aging (otherwise, biological age would be 100% defined by the age of HSCs), but at least place it among the major drivers of the process and pro-vide an explanation for our success in reliably determining biological age from blood test data. Rather, the identification of aging with the dynamics of a single organ-ism state variable, dFI, suggests a cross-talk in the form of continuous interactions between the organism components. dFI, hence emerges as a feature characterizing the organism as a whole, rather than representing a property of any particular subsystem.

The cooperative character of aging in the model im-plies that there is no specific subsystems tracking time or age in an animal. The age-dependent chances appear in a self-consistent manner by strong non-linear inter-actions between physiological compartments. Formally, this is expressed by representing the aging organism as an autonomous (or time-invariant) dynamical system having no designated subsystem for tracking time. Accordingly, we expected no physiological indices may depend on age of the animals explicitly, only implicitly via dependence on the collective variable, dFI. That is why, we believe, the analysis of dFI properties revealed that in addition to the trivial dependence on CBC features, which were directly involved in dFI calculation, the dFI was strongly correlated with certain measures of frailty, also known as hallmarks of aging. These include grip strength, body weight, RDW, and markers of inflammation such as CRP and KC (IL-8). dFI also correlated well with p16-luciferase flux, a proxy for the number of senescent cells in aged mice. We observed a very high degree of concordance between the dFI and the physio-logical frailty index (PFI), which is a combination of a much wider range of analyses than CBC, including physical fitness, cardiovascular health and biochemistry.

The dFI increased at a characteristic doubling rate of 0.022 per week, that is, in line with our theoretical prediction, comparable with the mortality rate doubling time in the species. Also, in the cross-sectional dataset the dFI saturated at a limiting value at the age corresponding to the average lifespan in the group. However, we observed that the dFI ceiling corresponds to the dFI levels in cohorts of animals scheduled for euthanasia due to morbid conditions under current laboratory protocols, which is as close to death as animals could possibly be in a modern laboratory. Therefore, we conclude that further dFI increments are incompatible with survival. It is thus dynamics of the organism state defining the unconstrained growth of dFI fueled by the dynamic instability of the organism state is the ultimate cause of death in aging mice. In [18], we explained that the exponential dFI acceleration is a signature of the linear dynamics in the weak coupling limit. At the maximum dFI level, the inevitably present non-linear effects take over and further evolution of the organism state occurs on much shorter time scales and lead to a complete disintegration of the organism.

The effects of non-linearity can be neglected nearly al-ways in the course of the life of an animal, if the dimensionless parameter expressing the animal lifespan in units of the mortality rate doubling time is small. Given the observed dFI doubling rates, we infer that the corresponding ratio is of the order of two, which is hardly large, and hence, non-linear corrections to the dynamics of the order parameter, dFI, should not be very small. Therefore, our linear AR model is only a reasonable approximation. We therefore believe that better performing dFI variants could be obtained by allowing for higher rank AR models, possibly including the effects of mode coupling with dFI.

The deep artificial neural network applied here also belongs to the class of unsupervised algorithms. It is re-markable that we used neither the remaining lifespan nor even the chronological age of the animals to infer dFI. This was possible, in principle, since by having a very specific model of the aging process, we were able to use longitudinal aging trajectories of individual animals for training. Due to the ability to obtain meaningful description of aging in the data without health or lifespan labels, the proposed method should be particularly useful for analysis of large longitudinal datasets from recently introduced sensors (such as wearable devices) often without any clinical and/or survival follow-up information avail-able.

Aging manifests itself as slow deviations of the organ-ism state from its initial state and can be tracked by measuring dFI. Our analysis shows that that the underlying organism state regulatory network in mice is dynamically unstable, and hence the organism state cannot relax to any equilibrium value after a perturbation. Formally this is expressed by strong auto-correlations of dFI over ex-tended periods of time. It is therefore likely that the effects of short treatments should persist until the end of life, whereas the effects of such treatments could be detected in short experiments involving longitudinal dFI measurements over a few months' time.

The dynamic character of dFI implies that most of the organism state changes associated with aging are in fact reversible. We therefore expect that further investigation of the longitudinal dynamics of physiological state variables and the associated biomarkers of aging and frailty could eventually lead to cost- and time-efficient clinical trials of upcoming anti-aging therapeutics.

Materials and Methods

A. Datasets

The training data set of CBC features was prepared from the nine data sources available in the Mouse Phenome Database (MPD) [13, 14]. List of the included sources is presented in Table S2 together with a statistic on animal number group by sex and age cohorts. Our model was trained using the best overlap of available CBC features from all sources. The final list contained 12 CBC features: granulocytes differential (GR %), granulocytes count (GR), hemoglobin (HB), hematocrit (HCT %), lymphocyte differential (LY %), lymphocyte count (LY), mean corpuscular hemoglobin content (MCH), mean hemoglobin concentration (MCHC), mean corpuscular volume (MCV), platelet count (PLT), red blood cell count (RBC) and white blood cell count (WBC). In the case of data source had no granulocytes measurements, it was retrieved using formulas:

GR=WBC−LY−MO

GR %=100−LY %−MO %

All animals with the missing data were excluded from the training.

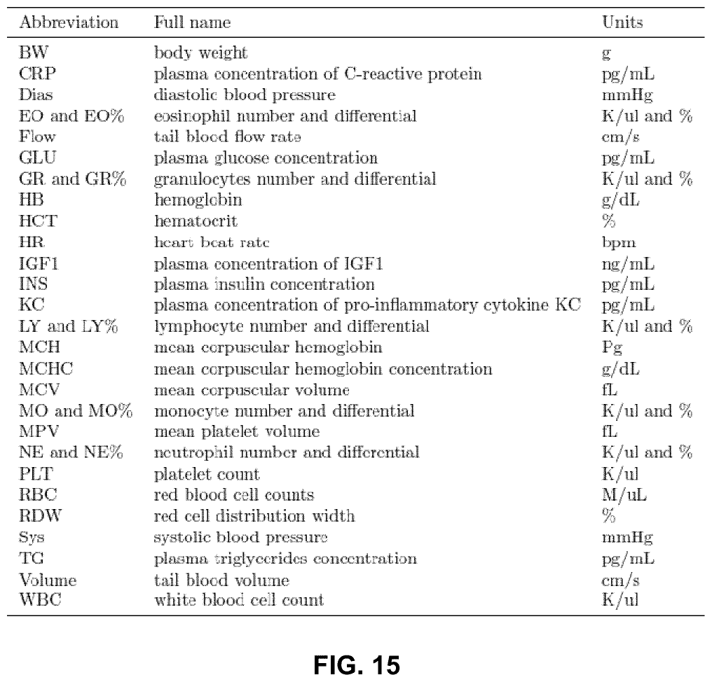

The list of all abbreviations is shown in FIG. 15.

B. Animals

Four-to-five week-old NIH Swiss male and female mice were obtained from Charles River Laboratories (Wilmington, Mass.) and were allowed to age within the Roswell Park Comprehensive Cancer Center (RPCCC) animal facility. Blood samples were obtained at different ages as part of creating of the Physiological Frailty Index (PFI) as previously described (REF). p16/INK4a-LUC mice (p16-Luc) were obtained from the N. Sharpless laboratory at the University of North Carolina (Chapel Hill, N.C.). All animals were housed under 12:12 light:dark conditions (12 hours of light followed by 12 hours of darkness) at the Laboratory Animal Shared Resource at RPCCC. All animal experiments were approved by the Institutional Animal Care and Use Committee of Roswell Park Cancer Institute.

Dataset MA0071 was built in a cross-sectional experiment using male and female NIH Swiss mice. Blood was collected from male mice by cardiac puncture at 26 (n=20), 64 (n=20), 78 (n=20), 92 (n=20), and 136 (n=8) week old mice. Female age groups were rep-resented by 30 (n=20), 56 (n=20), 68 (n=20), 82 (n=20), 95 (n=20), 108 (n=20), and 136 (n=8) weeks of age.

Dataset MA0072 was obtained from a longitudinal experiment. Blood samples were collected through saphenous vein from male NIH Swiss mice at 66 (n=30), 81 (n=24), 94 (n=22), 109 (n=18), and 130 (n=11) weeks of age.

Dataset MA0073 includes blood samples collected from 97 male and 127 female mice of different ages when animals reached approved experimental endpoints and re-quire humane euthanasia. Whole blood cell analysis was performed in 20 μl of blood using Hemavet 950 Analyzer (Drew Scientific) according to manufacturer's protocol. For rapamycin treatment experiment 60-weeks-old C57BL/6J male mice were obtained from Jackson Laboratories (USA). Rapamycin was purchased from LC Lab-oratories (MA, USA). Rapamycin was administered daily at 12 mg/kg via oral gavage for 8 weeks. Control group was treated with vehicle (5% Tween-80, 5% PEG-400, 3% DMSO).

C. In Vivo Bioluminescence Imaging

Bioluminescence imaging was performed using an IVIS Spectrum imaging system (Caliper LifeSciences, Inc, Waltham, Mass.). p16/Ink4a-Luc+/− female mice were injected. intraperitoneally with D-Luciferin (150 mg/kg, Gold Biotechnology), 3 minutes later anesthetized with isoflurane and imaged using a 20-second integration time and medium binning. Data were quantified as the sum of photon flux recorded from both sides of each mouse using Living Image software (Perkin Elmer, Waltham, Mass.).

D. Dimensionality Reduction with PCA

Principal component analysis (PCA) was performed with Python [57] and Scikit-learn package [58]. First, we applied PCA transformation on the entire training dataset. However, the principal components were dominated by the difference of mice strains. Animals of the same strains were clustered on the plot of the first principal component against the second one. We removed strain difference by subtracting mean values of CBC features calculated for the earliest age available for the selected strain from values of CBC features of all animals for this strain. For the simplicity we restricted our analysis to 30 strains, which were presented in the Peters4 dataset.

E. Statistical Analysis of Mortality Data

The death records for animals linked with the MPD dataset Peters4 were also available in MPD as the dataset named Yuan2 [59]. The Spearman's rank correlation test was performed with Python and SciPy package [60]. The analysis was performed for two cohorts of mice. The first cohort included all animals from the Peters4 dataset with mortality data from Yuan2. The second cohort included animals from the Peters4 dataset with the measurements of body weight and IGF1 serum level taken from MPD dataset named Yuan1 [26].

F. Neural Network Structure

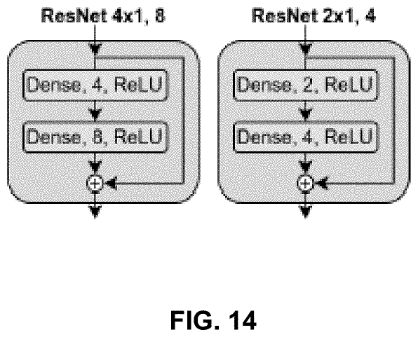

The neural network was designed to handle a specific problem: the disbalance of samples with longitudinal and cross-sectional measurements. As inputs, the network has three 12-dimensional vectors: one for the cross-sectional dataset, and two others for the longitudinal dataset corresponding to the present state and future state of a sample. Inputs pass through the encoder part of the auto-encoder block and then split up (see FIG. 13). Cross-sectional samples are directed to the decoder part, while longitudinal samples in the compressed representation are passed for the training autoregression part. Such data flow allows the auto-encoder to be deeper and train without overfitting by using more samples from a larger cross-sectional dataset. The auto-encoder has the architecture of a linear stack of fully connected dense layers and residual network blocks (ResNet) [61]. Dense layers have a trainable weight matrix W, bias vector b, and linear activation function by default. The ResNet block, shown in FIG. 14, is a stack of two dense layers with an activation function of rectified linear unit (ReLU)[62], input and output are linked by applying element-wise addition. ResNet blocks add nonlinear rectification transformations to the original input, helping to learn non-linear transformations. To prevent overfitting, we applied L2 regularization of factor 0.01 to model weights W.

FIG. 15 is a table listing all abbreviations used in this disclosure.

FIG. 16 is a table comprising a complete list of datasets used for training models disclosed herein.

FIG. 17 is a table describing the various test datasets used.

FIG. 18 is a table illustrating reconstruction error (root-mean square error, RMSE) and coefficient of determination, R², of the autoencoder calculated for each CBC feature in the training set.

FIG. 19 is a table illustrating reconstruction error (root-mean square error, RMSE) and coefficient of determination, R², of the autoencoder calculated for each CBC feature in the test set.

FIG. 20 illustrates principal component analysis (PCA) of the MPD data (including young animals). The graphs represent the average of the PC scores in subsequent age groups. The inset shows that the variance for all PC scores increase with age.

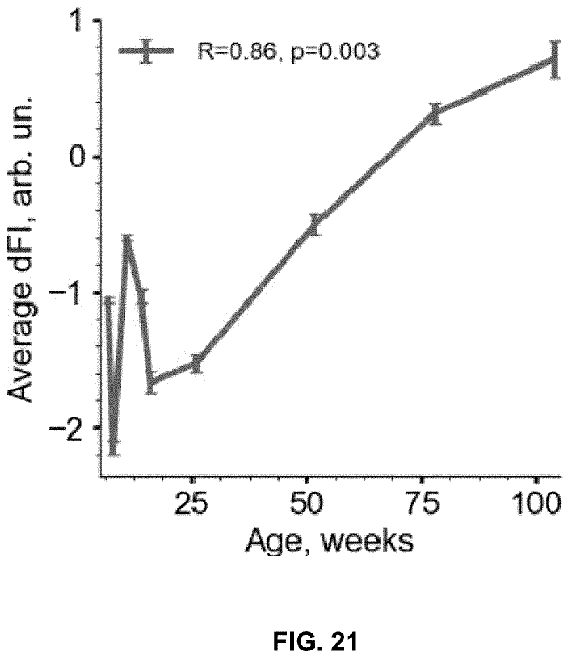

FIG. 21 is a graph illustrating the growth of age cohort average dFI with age in the training dataset.

FIG. 22 illustrates clustering of CBC features and dFI score in the test dataset. The colors represent the Pearson's correlation coefficient (absolute value) as indicated by the scale on the right side of the figure.

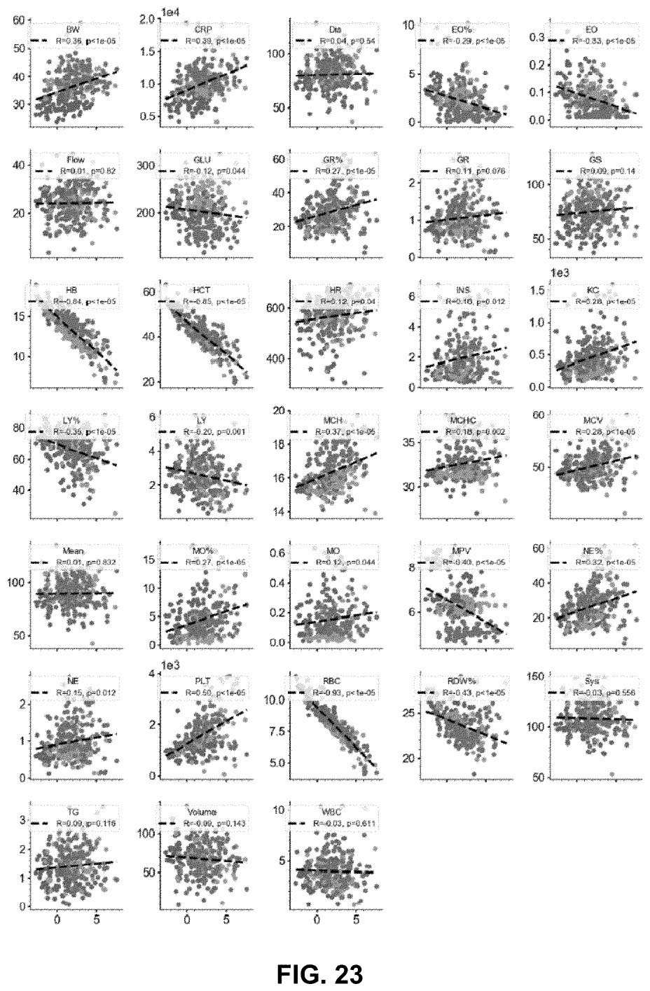

FIG. 23 illustrate correlations between dFI and other biological markers. The colors represent the following datasets: blue represents females in MA0071, orange represents males in MA0071, and green represents males in MA0072.

FIG. 24 illustrates correlations between dFI and CBC parameters in the Peters4 dataset. Colors from blue to red represent age of animals, where blue is age of 26 weeks and red is age of 104 weeks.

FIGS. 25A and 25B illustrate correlations between dFI and CBC parameters in the Peters4 dataset shown for cohort of mice of same strain and sex.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved. Finally, terms of degree such as “substantially”, “about” and “approximately” as used herein mean a reasonable amount of deviation (e.g., a deviation of up to ±0.1%, ±1%, ±5%, or ±10%, as such variations are appropriate) from the specified value such that the end result is not significantly or materially changed.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

### REFERENCES

[1] T. L. Gruenewald, T. E. Seeman, C. D. Ryff, A. S. Kar-lamangla, and B. H. Singer, Proc. Natl. Acad. Sci. U.S.A 103, 14158 (2006).
[2] M. Moeller, M. Hirose, S. Mueller, C. Roolf, S. Baltrusch, S. Ibrahim, C. Junghanss, O. Wolkenhauer, R. Jaster, R. Kohling, M. Kunz, M. Tiedge, P. N. Schofield, and G. Fuellen, Aging Cell 13, 729 (2014).
[3] P. Sebastiani, B. Thyagarajan, F. Sun, N. Schupf, A. B. Newman, M. Montano, and T. T. Perls, Aging Cell 16, 329 (2017).
[4] A. Burkle, M. Moreno-Villanueva, J. Bernhard, M. Blasco, G. Zondag, J. H. Hoeijmakers, O. Toussaint, B. Grubeck-Loebenstein, E. Mocchegiani, S. Collino, E. S. Gonos, E. Sikora, D. Gradinaru, M. Dolle, M. Salmon, P. Kristensen, H. R. Griffiths, C. Libert, T. Grune, N. Breusing, A. Simm, C. Franceschi, M. Capri, D. Talbot, P. Caiafa, B. Friguet, P. E. Slag-boom, A. Hervonen, M. Hurme, and R. Aspinall, Mech. Ageing Dev. 151, 2 (2015).
[5] S. Gonzalo, Epigenetic alterations in aging (2010).
[6] S. Horvath, Genome Biol. 14, R115 (2013).
[7] S. Horvath, C. Pirazzini, M. G. Bacalini, D. Gentilini, A. M. Di Blasio, M. Delledonne, D. Mari, B. Arosio, D. Monti, G. Passarino, F. De Rango, P. D'Aquila, C. Giuliani, E. Marasco, S. Collino, P. Descombes, P. Garagnani, and C. Franceschi, Aging (Albany. NY). 7, 1159 (2015).
[8] R. E. Marioni, S. Shah, A. F. McRae, B. H. Chen, E. Col-icino, S. E. Harris, J. Gibson, A. K. Henders, P. Red-mond, S. R. Cox, A. Pattie, J. Corley, L. Murphy, N. G. Martin, G. W. Montgomery, A. P. Feinberg, M. D. Fallin, M. L. Multhaup, A. E. Jaffe, R. Joehanes, J. Schwartz, A. C. Just, K. L. Lunetta, J. M. Murabito, J. M. Starr, S. Horvath, A. A. Baccarelli, D. Levy, P. M. Visscher, N. R. Wray, and I. J. Deary, Genome Biol. 16, 25 (2015).
[9] R. L. Sprott and B. E. Eleftheriou, Gerontology 20, 155 (1974).
[10] T. E. Seeman, L. F. Berkman, P. A. Charpentier, D. G. Blazer, M. S. Albert, and M. E. Tinetti, Journals Geron-tol. Ser. A Biol. Sci. Med. Sci. 50A, M177 (1995).
[11] T. V. Pyrkov, E. Getmantsev, B. Zhurov, K. Avchaciov, M. Pyatnitskiy, L. Menshikov, K. Khodova, A. V. Gud-kov, and P. O. Fedichev, Aging (Albany. NY). 10, 2973 (2018).
[12] H. Shoji, K. Takao, S. Hattori, and T. Miyakawa, Mol. Brain 9, 10.1186/s13041-016-0191-9 (2016).
[13] M. A. Bogue, L. L. Peters, B. Paigen, R. Korstanje, R. Yuan, C. Ackert-Bicknell, S. C. Grubb, G. A. Churchill, and E. J. Chesler, The Journals of Gerontol-ogy: Series A 71, 170 (2016).
[14] M. A. Bogue, S. C. Grubb, D. O. Walton, V. M. Philip, G. Kolishovski, T. Stearns, M. H. Dunn, D. A. Skelly, B. Kadakkuzha, G. Tehennepe, G. Kunde-Ramamoorthy, and E. J. Chesler, Nucleic Acids Res. 10.1093/nar/gkx1082 (2018).
[15] K. E. O'Connell, A. M. Mikkola, A. M. Stepanek, A. Ver-net, C. D. Hall, C. C. Sun, E. Yildirim, J. F. Staropoli, J. T. Lee, and D. E. Brown, Comp. Med. 65, 96 (2015).
[16] D. Krotov, J. O. Dubuis, T. Gregor, and W. Bialek, Proc. Natl. Acad. Sci. 111, 3683 (2014).
[17] H. Haken, Phys. Astron. online Libr. (Springer, Berlin, Heidelberg, 2004).
[18] D. Podolskiy, I. Molodtcov, A. Zenin, V. Kogan, L. I. Menshikov, R. J. S. Reis, and P. O. Fedichev, arXiv Prepr. arXiv1502.04307 (2015).
[19] M. P. Antoch, M. Wrobel, K. K. Kuropatwinski, I. Gitlin, K. I. Leonova, I. Toshkov, A. S. Gleiberman, A. D. Hut-son, O. B. Chernova, and A. V. Gudkov, Aging (Albany N.Y.) 9, 615 (2017).
[20] K. Pearson, London, Edinburgh, Dublin Philos. Mag. J. Sci. 2, 559 (1901).
[21] H. Hotelling, J. Educ. Psychol. 24, 417 (1933).
[22] I. T. Jollife and J. Cadima, Principal component analysis: A review and recent developments (2016).
[23] M. Scheffer, J. E. Bolhuis, D. Borsboom, T. G. Buch-man, S. M. W. Gijzel, D. Goulson, J. E. Kammenga, B. Kemp, I. A. van de Leemput, S. Levin, C. M. Mar-tin, R. J. F. Melis, E. H. van Nes, L. M. Romero, and M. G. M. Olde Rikkert, Proc. Natl. Acad. Sci. U.S.A 115, 11883 (2018).
[24] B. G. Hughes and S. Hekimi, Genetics 204, 905 (2016).
[25] R. Yuan, Q. Meng, J. Nautiyal, K. Flurkey, S. W. Tsaih, R. Krier, M. G. Parker, D. E. Har-rison, and B. Paigen, Proceedings of the Na-tional Academy of Sciences 109, 8224 (2012), http://www.pnas.org/content/109/21/8224.full.pdf.
[26] R. Yuan, S.-W. Tsaih, S. B. Petkova, C. M. De Evsikova, S. Xing, M. A. Marion, M. A. Bogue, K. D. Mills, L. L. Peters, C. J. Bult, C. J. Rosen, J. P. Sundberg, D. E. Harrison, G. A. Churchill, and B. Paigen, Aging Cell 8, 277 (2009).
[27] T. S. Perlstein, J. Weuve, M. A. Pfeffer, and J. A. Beck-man, Arch. Intern. Med. 169, 588 (2009).
[28] K. V. Patel, R. D. Semba, L. Ferrucci, A. B. Newman, L. P. Fried, R. B. Wallace, S. Bandinelli, C. S. Phillips, B. Yu, S. Connelly, M. G. Shlipak, P. H. Chaves, L. J. Launer, B. Ershler, T. B. Harris, D. L. Longo, and J. M. Guralnik, Journals Gerontol. —Ser. A Biol. Sci. Med. Sci. 65 A, 258 (2010).
[29] M. Baggiolini, Chemokines and leukocyte traffic (1998).
[30] T. B. Harris, L. Ferrucci, R. P. Tracy, M. C. Corti, S. Wa-cholder, W. H. Ettinger, H. Heimovitz, H. J. Cohen, and R. Wallace, Am. J. Med. 106, 506 (1999).
[31] N. R. Sproston and J. J. Ashworth, Role of C-reactive protein at sites of inflammation and infection (2018).
[32] T. Kuilman, C. Michaloglou, W. J. Mooi, and D. S. Peeper, Genes Dev. 24, 2463 (2010).
[33] J. M. van Deursen, Nature 509, 439 (2014).
[34] B. M. Hall, V. Balan, A. S. Gleiberman, E. Strom, P. Krasnov, L. P. Virtuoso, E. Rydkina, S. Vujcic, K. Balan, I. Gitlin, K. Leonova, A. Polinsky, O. B. Cher-nova, and A. V. Gudkov, Aging (Albany. NY). 8, 1294 (2016).
[35] W. Y. Kim and N. E. Sharpless, Cell 127, 265 (2006).
[36] C. E. Burd, J. A. Sorrentino, K. S. Clark, D. B. Darr, J. Krishnamurthy, A. M. Deal, N. Bardeesy, D. H. Cas-trillon, D. H. Beach, and N. E. Sharpless, Cell 152, 340 (2013).
[37] J. E. Wilkinson, L. Burmeister, S. V. Brooks, C.-C. Chan, S. Friedline, D. E. Harrison, J. F. Hejtmancik, N. Nadon, R. Strong, L. K. Wood, M. A. Woodward, and R. A. Miller, Aging Cell 11, 675 (2012).
[38] W. R. Swindell, Journals Gerontol. —Ser. A Biol. Sci. Med. Sci. 72, 1024 (2017).
[39] R. A. Miller, D. E. Harrison, C. M. Astle, E. Fernandez, K. Flurkey, M. Han, M. A. Javors, X. Li, N. L. Nadon, J. F. Nelson, S. Pletcher, A. B. Salmon, Z. D. Sharp, S. Van Roekel, L. Winkleman, and R. Strong, Aging Cell 13, 468 (2014).
[40] E. Nakamura, K. Miyao, and T. Ozeki, Mech. Ageing Dev. 46, 1 (1988).
[41] P. Klemera and S. Doubal, Mech. Ageing Dev. 127, 240 (2006).
[42] E. Balleza, E. R. Alvarez-Buylla, A. Chaos, S. Kauffman, I. Shmulevich, and M. Aldana, PLoS One 3, e2456 (2008).
[43] V. Kogan, I. Molodtsov, L. I. Menshikov, R. J. S. Reis, and P. Fedichev, Scientific reports 5, 13589 (2015).
[44] L. D. Landau and E. M. Lifshitz, Course Theor. Phys. 5 (1980).
[45] I. M. Johnstone and A. Y. Lu, Journal of the American Statistical Association 104, 682 (2009).
[46] A. Mardt, L. Pasquali, H. Wu, and F. No'e, Nat. Com-mun. 9, 5 (2018).
[47] B. Lusch, J. N. Kutz, and S. L. Brunton, Nat. Commun. 9, 4950 (2018).
[48] B. Hofmann, Young Blood Rejuvenates Old Bodies: A Call for Reflection when Moving from Mice to Men (2018).
[49] I. Beerman, C. Bock, B. S. Garrison, Z. D. Smith, H. Gu, A. Meissner, and D. J. Rossi, Cell Stem Cell 12, 413 (2013).
[50] C. Franceschi, M. Bonaf'e, S. Valensin, F. Olivieri, M. De Luca, E. Ottaviani, and G. De Benedictis, Ann. N. Y. Acad. Sci. 908, 244 (2006).
[51] T. Fulop, G. Dupuis, J. M. Witkowski, and A. Larbi, The role of immunosenescence in the development of age-related diseases (2016).
[52] E. Fuentes, M. Fuentes, M. Alarc'on, and I. Palomo, An. Acad. Bras. Cienc. 89, 285 (2017).
[53] G. Pawelec, Biogerontology 18, 717 (2017).
[54] S. N. Crooke, I. G. Ovsyannikova, G. A. Poland, and R. B. Kennedy, Immun Ageing 16, 10.1186/s12979-019-0164-9 (2019).
[55] W. W. Pang, E. A. Price, D. Sahoo, I. Beerman, W. J. Maloney, D. J. Rossi, S. L. Schrier, and I. L. Weissman, Proc. Natl. Acad. Sci. U.S.A 108, 20012 (2011).
[56] R. A. Signer, E. Montecino-Rodriguez, O. N. Witte, J. McLaughlin, and K. Dorshkind, Blood 110, 1831 (2007).
[57] T. E. Oliphant, Comput. Sci. Eng. 9, 10 (2007).
[58] F. Pedregosa, G. Varoquaux, A. Gramfort, V. Michel, B. Thirion, O. Grisel, M. Blondel, P. Prettenhofer, R. Weiss, V. Dubourg, J. Vanderplas, A. Passos, D. Cournapeau, M. Brucher, M. Perrot, and E. Duchesnay, J. Mach. Learn. Res. 12, 2825 (2011).
[59] R. Yuan, Q. Meng, J. Nautiyal, K. Flurkey, S.-W. Tsaih, R. Krier, M. G. Parker, D. E. Harrison, and B. Paigen, Proc. Natl. Acad. Sci. 109, 8224 (2012).
[60] P. Virtanen, R. Gommers, T. E. Oliphant, M. Haber-land, T. Reddy, D. Cournapeau, E. Burovski, P. Pe-terson, W. Weckesser, J. Bright, S. J. van der Walt, M. Brett, J. Wilson, K. Jarrod Millman, N. Mayorov, A. R. Nelson, E. Jones, R. Kern, E. Larson, C. J. Carey, b. Polat, Y. Feng, E. W. Moore, J. Vander-Plas, D. Laxalde, J. Perktold, R. Cimrman, I. Henriksen, E. Quintero, C. R. Harris, A. M. Archibald, A. H. Ribeiro, F. Pedregosa, P. van Mulbregt, arXiv e-prints, arXiv:1907.10121 (2019), arXiv:1907.10121 [cs.MS].
[61] K. He, X. Zhang, S. Ren, and J. Sun, in Proc. IEEE Comput. Soc. Conf. Comput. Vis. Pattern Recognit., Vol. 2016-Decem (IEEE Computer Society, 2016) pp. 770-778, arXiv:1512.03385.
[62] V. Nair and G. E. G. E. G. E. Hinton, in Proc. 27th Int. Conf. Int. Conf. Mach. Learn., ICML'10 (Omnipress, USA, 2010) pp. 807-814.

We claim:

1. A method for determining a biological age of a mammal, comprising:
inputting values of at least six health parameters from the mammal; and
performing a set of further steps, wherein the set of further steps is selected from the following:
set 1: determining the biological age of the mammal by calculating the biological age of the mammal using an algorithm comprising multiple mathematical operations, wherein the algorithm is defined by a Pearson correlation coefficient higher than 0.5, wherein the Pearson correlation coefficient is determined by:
a. calculating a first biological age of a plurality of mammals of the same phenotype at a first time represented by a first vector X;
b. calculating a second biological age of the plurality of mammals of the same phenotype at a second time represented by a second vector Y; and
c. determining the Pearson correlation coefficient between vectors X and Y;
set 2: wherein, such inputting values of at least six of health parameters of the mammals is done into computer, a calculation of biological age by application of algorithm comprising performance of multiple mathematical operations, at least multiplication by matrix and summation of vectors to inputted values of health parameters (those values of health parameters that were inputted according the previous step), wherein said biological age is a single number (score), and the said algorithm has at least the following features:
1) if one will use the said algorithm to determine scores using values of the same health parameters of at least 50 of mammals of the same phenotype, wherein each individual animal must have an unique identification label (for example: A1 for animal 1 and A2 for animal 2),
2) repeat clause (a) with the same mammals but health parameters are obtained from the same individual animals not later than period of 10% of such mammals average lifespan after the date of obtaining health parameters from the same individual animal in clause (a), and
3) than a Pearson correlation coefficient between vectors X and Y will have value higher than of 0.5, if Pearson correlation calculated in the following way: one should take values of the score for each animal from clause (a) and form a vector X, then take values of the score from clause (b) and form the vector Y, wherein to construct both vectors X and Y the scores should be placed to keep ordering of identification labels (for example, X=[score^{t1}ₐ₁, score^{t1}ₐ₂, . . . , score^{t1}ₐ₅₀) and Y=[score^{t2}ₐ₁, score^{t2}ₐ₂, . . . , score^{t2}ₐ₅₀);
set 3: determining the biological age of the mammal by calculating the biological age of the mammal using an algorithm comprising multiple mathematical operations, wherein the algorithm predicts scores which order animals by their survival time, where in the spearman's rank-order correlation between such scores and real survival times should be negative number with the corresponding p-values lower than 0.05.

2. The method of claim 1, wherein the Pearson correlation coefficient is selected from the group: higher than of 0.55, higher than of 0.6, in the range from 0.5 to 0.7, in the range from 0.6 to 0.8, in the range from 0.5 to 0.9, in the range from 0.5 to 0.99, in the range from 0.55 to 0.99, higher than of 0.7, higher than of 0.8, higher than of 0.9, higher than of 0.95, higher than of 0.99.

3. The method of claim 1, wherein Spearman's rank-order correlation p-values 1 is selected from the following group: lower than 0.03, lower than 0.01, lower than 0.005, lower than 0.003, lower than 0.001, lower than 0.0005, lower than 0.0003, lower than 0.0001, lower than 0.00005, lower than 0.00003, lower than 0.00001, lower than 0.000001, lower than 0.0000001, in the range from 0.05 to 0.0000001, in the range from 0.01 to 0.000001, in the range from 0.001 to 0.00001.

4. The method of claim 1, wherein the p-value is selected from the following group for a corresponding number of mammals:
N p-value
for 20 mammals—lower than 0.05,
for 20 mammals—lower than 0.03,
for 20 mammals—lower than 0.01,
for 20 mammals—in the range from 0.04 to 0.01,
for 20 mammals—in the range from 0.04 to 0.001,
for 30 mammals—lower than 0.02,
for 50 mammals—lower than 0.01,
for 50 mammals—lower than 0.001,
for 100 mammals—lower than 0.001
for 150 mammals—lower than 1E-05,
for >200 mammals—lower than 1E-6.

5. The method of claim 1, wherein the biological age is a score.

6. The method of claim 1, wherein the biological age is a score selected from a single value or number.

7. The method of claim 1, wherein the mathematical operations comprise multiplication of matrices and summation of vectors of inputted values of the health parameters.

8. The method of claim 1, further comprising determining the algorithm using a neural network architecture.

9. The method of claim 8, wherein determining the algorithm comprises:
obtaining health parameters and corresponding ages from a plurality of mammals; and
inputting the health parameters and the corresponding ages of the mammals into an autoencoder of the neural network architecture.

10. The method of claim 9, wherein the health parameters are determined based on blood parameters.

11. The method of claim 8, wherein the mammals are alive and selected from one of the following: mice, humans, dogs, cats, non-human primates, rats, guinea pigs, rabbits, hamsters, sheep, gerbils, bats, ferrets, chinchillas, goats, and horses.

12. The method of claim 9, wherein health parameters are selected from the following blood parameters: granulocytes differential (gr, %), granulocytes count (gr, K/l), hemoglobin (hb, g/dl), hematocrit (hct, %), lymphocyte differential (ly, %), lymphocyte count (ly, K/l), mean corpuscular hemoglobin content (mch, pg), mean hemoglobin concentration (mchc, g/dl), mean corpuscular volume (mcv, fl), platelet count (plt, K/l), red blood cell count (rbc, M/l) and white blood cell count (wbc, K/l).

13. The method of claim 12, wherein granulocytes are unavailable, it is calculated using the following formulas:
gr(K/l)=wbc(K/l)−ly(K/l)−mo(K/l)gr(%)=100−ly(%)−mo(%)

14. The method of claim 10, wherein the health parameters are selected from a complete blood count.

15. The method of claim 9, wherein the health parameters comprise HB (g/dL), LY (K/μL), MCH (Pg), MCHC (g/dL), MCV(fL), MO (K/μL), PLT, RBC (M/uL), WBC (K/μL).

16. The method of claim 1, wherein the determination of biological age comprises following steps:
1) subtract the reference mean value (column MEAN in the table) of each test;
2) multiply by the coefficient from column COEF;
MEAN,COEF
HB (g/dL),14.7810810811,−0.324994418476
LY (K/μL),6.78821787942,−0.0403357974256
MCH (Pg),15.2156964657,−0.305640352983
MCHC (g/dL),33.18497921,0.0243410007583 MCV(fL),45.8556652807,−0.071912079313
MO (K/μL),0.187391325364,2.99337099222 MPV,5.82976611227,−0.0622717180147 PLT,1258.6456341,0.00122980926892
RBC (M/uL),9.74016632017,−0.227470069201
WBC (K/μL),8.83614345114,0.0437124309324

17. The method of claim 1, wherein the health parameters are selected from at least one of the following: complete blood count, basic metabolic panel, comprehensive metabolic panel, lipid panel, liver panel, thyroid stimulating hormone, Hemoglobin A1C, and c-reactive protein.

18. The method of claim 1, wherein the health parameters are selected from the group: Glucose, serum (mg/dL); Creatinine (mg/dL); Lactate dehydrogenase LDH (U/L); Uric acid (mg/dL); Blood lead (ug/dL); Homocysteine(μmol/L); Vitamin A (ug/dL); Fasting Glucose (mg/dL); GGT: SI (U/L); Total cholesterol (mg/dL); Vitamin E (ug/dL); Chloride: SI (mmol/L); AST: SI (U/L); Sodium: SI (mmol/L); PCB180 (ng/g); Cholesterol (mg/dL); PCB170 (ng/g); Alkaline phosphatase (U/L); PCB180 Lipid Adjusted; Oxychlordane Lipid Adjusted; 3,3′,4,4′,5,5′-hexachlorobiphenyl (hxcb) (fg/g); PCB74 (ng/g); PCB170 Lipid Adjusted; Triglycerides (mg/dL); PCB153 (ng/g); Oxychlordane (ng/g); PCB74 Lipid Adjusted; Monocyte percent (%); Ferritin (ng/mL); 3,3′,4,4′,5,5′-hexachlorobiphenyl (hxcb) Lipid Adjusted; 2,3,4,7,8-Pentachlorodibenzofuran (pncdf) (fg/g); Methylmalonic acid (umol/L); PCB153 Lipid Adjusted; PCB187 (ng/g); 2,3,4,7,8-Pentachlorodibenzofuran (pncdf) Lipid Adjusted; PCB156 (ng/g); White blood cell count: SI; PCB187 Lipid Adjusted; 1,2,3,6,7,8-Hexachlorodibenzo-p-dioxin (hxcdd)(fg/g); Trans-nonachlor Lipid Adjusted; PCB138 (ng/g); 4-pyridoxic acid (nmol/L); Potassium: SI (mmol/L); Trans-nonachlor (ng/g); 1,2,3,6,7,8-Hexachlorodibenzo-p-dioxin (hxcdd) Lipid Adjusted; PCB138 Lipid Adjusted; PCB118 (ng/g); PCB156 Lipid Adjusted; PCB118 Lipid Adjusted; Mean cell volume (IL); PCB146 (ng/g); Blood cadmium (ug/L); Two hour oral glucose tolerance (OGTT) (mg/dL); Folate, serum (ng/mL); PCB194 Lipid Adjusted; PCB194 (ng/g); Hematocrit (%); 1,2,3,4,7,8-Hexachlorodibenzofuran (hcxdf) (fg/g); Perfluorohexane sulfonic acid (ug/L); RBC folate (nmol/L); PCB99 (ng/g); p,p′-DDE (ng/g); p,p′-DDE Lipid Adjusted; Total Serum Foalte (nmol/L); PCB146 Lipid Adjusted; PCB196 Lipid Adjusted; PCB196 (ng/g); 1,2,3,4,6,7,8,9-Octachlorodibenzo-p-dioxin (ocdd) (fg/g); PCB183 (ng/g); Perfluorooctane sulfonic acid; 3,3′,4,4′,5-Pentachlorobiphenyl (pncb) (fg/g); trans-lycopene (ug/dL); 1,2,3,7,8-Pentachlorodibenzo-p-dioxin (pncdd) (fg/g); 1,2,3,4,6,7,8-Heptachlororodibenzo-p-dioxin (hpcdd) (fg/g); 3,3′,4,4′,5-Pentachlorobiphenyl (pncb) Lipid Adjusted; 1,2,3,4,7,8-Hexachlorodibenzofuran (hcxdf) Lipid Adjusted; 1,2,3,6,7,8-Hexachlorodibenzofuran (hxcdf) (fg/g); PCB99 Lipid Adjusted; Triiodothyronine (T3), free (pg/mL); 1,2,3,4,6,7,8,9-Octachlorodibenzo-p-dioxin (ocdd) Lipid Adjusted; a-Tocopherol (ug/dL); Blood o-Xylene Result; Beta-hexachlorocyclohexane Lipid Adjusted; Plasma glucose: SI (mmol/L); 1,2,3,7,8-Pentachlorodibenzo-p-dioxin (pncdd) Lipid Adjusted; Parathyroid Hormone (Elecys method) pg/mL; Beta-hexachloro-cyclohexane (ng/g); 1,2,3,4,6,7,8-Heptachlororodibenzo-p-dioxin (hpcdd) Lipid Adjusted; PCB105 (ng/g); PCB177 (ng/g); Hemoglobin (g/dL); Heptachlor Epoxide (ng/g); Perfluorooctanoic acid; Heptachlor Epoxide Lipid Adjusted; 1,2,3,6,7,8-Hexachlorodibenzofuran (hxcdf) Lipid Adjusted; PCB183 Lipid Adjusted; 2,3,7,8-Tetrachlorodienzo-p-dioxin (tcdd) (fg/g); Vitamin B12, serum (pg/mL); cis-b-carotene (ug/dL); Cotinine (ng/mL); 1,2,3,7,8,9-Hexachlorodibenzo-p-dioxin (hxcdd) (fg/g); Triglyceride (mg/dL); p,p′-DDT (ng/g); Triiodothyronine (T3), total (ng/dL); PCB105 Lipid Adjusted; 1,2,3,4,7,8-Hexachlorodibenzo-p-dioxin (hxcdd)(fg/g); Mean cell hemoglobin (pg); Dieldrin (ng/g); Folate, RBC (ng/mL RBC); Aldrin; trans-b-carotene (ug/dL); Eosinophils percent (%); Endrin; Bone alkaline phosphotase (ug/L); PCB199 Lipid Adjusted; 1,2,3,4,7,8-Hexachlorodibenzo-p-dioxin (hxcdd) Lipid Adjusted; 1,2,3,7,8,9-Hexachlorodibenzo-p-dioxin (hxcdd) Lipid Adjusted; Dieldrin Lipid Adjusted; p,p′-DDT Lipid Adjusted; Segmented neutrophils percent (%); 2,3,7,8-Tetrachlorodienzo-p-dioxin (tcdd) Lipid Adjusted; Retinyl stearate (ug/dL); PCB151 (ng/g); PCB149 (ng/g); Perfluorononanoic acid (ug/L); PCB177 Lipid Adjusted; PCB178 Lipid Adjusted; PCB209 (ng/g); PCB178 (ng/g); 5-Methyl THF (nmol/L); PCB209 Lipid Adjusted (ng/g); C-peptide (nmol/L) in SI units; Platelet count (%) SI; Blood Bromodichloromethane Result; Total iron binding capacity (ug/dL); Red cell distribution width (%); Blood Chloroform Result; Glycidamide (pmoL/G Hb); Testosterone total (ng/dL); Hexachlorobenzene (ng/g); Apolipoprotein (B) (mg/dL); ALT: SI (U/L); 25-hydroxyvitamin D2+D3; PCB206 Lipid Adjusted; Follicle stimulating hormone (mIU/mL); Basophils percent (%); 2-(N-Methyl-perfluorooctane sulfonamido) acetic acid (ug/L); Vitamin B6 (Pyridoxal 5′-phosphate) test results (nmol/L); Pyridoxal 5′-phosphate (nmol/L); total Lycopene (ug/dL); Blood Methyl t-Butyl Ether (MTBE) Result; Helicobacter pylori (ISR); PCB167 Lipid Adjusted; Mirex (ng/g); Luteinizing hormone (mIU/mL); Blood manganese (ug/L); Mean cell hemoglobin concentration (g/dL); PCB128 (ng/g); a-Cryptoxanthin (ug/dL); Thyroxine, free (ng/dL); cis-Lycopene (ug/dL); Thyroid stimulating hormone (uIU/mL); PCB172 Lipid Adjusted; Blood mercury, total (ug/L); Inorganic mercury, blood (ug/L); 2,2′,4,4′,5,5′-hexabromobiphenyl (pg/g); Vitamin C (mg/dL); Blood m-/p-Xylene Result; PCB167 (ng/g); Mercury, methyl (ug/L); Combined Lutein/zeaxanthin (ug/dL); 2,2′,4,4′,5,6′-hexabromodiphenyl ether (pg/g); Folic acid, serum (nmol/L); Acrylamide (pmoL/G Hb); 2,2′,4,4′,5,5′-hexabromobiphenyl lipid adjusted (ng/g); 2,3,4,6,7,8,-Hexchlorodibenzofuran (hxcdf) (fg/g); total b-Carotene (ug/dL); 25-hydroxyvitamin D3 (nmol/L); Perfluoroundecanoic acid (ug/L); Protoporphyrin (ug/dL RBC); PCB206 (ng/g); PCB157 Lipid Adjusted; Phytofluene (ug/dL); Aldrin Lipid Adjusted; epi-25-hydroxyvitamin D3 (nmol/L); PCB172 (ng/g); PCB66 (ng/g); Endrin Lipid Adjusted; a-carotene (ug/dL); Trans 9, trans 12-octadienoic acid (uM); PCB28 (ng/g); Pefluorodecanoic acid (ug/L); Lymphocyte percent (%); Thyroid stimulating hormone (IU/L); 1,2,3,4,6,7,8-Heptachlorodibenzofuran (hpcdf) (fg/g); Hexachlorobenzene Lipid Adjusted; Mirex Lipid Adjusted; Total dust weight (mg); Insulin: SI (pmol/L); Sieved dust weight (mg); Serum Selenium (ug/L); Lutein (ug/dL); Blood Nitromethane (pg/mL); Gamma-hexachlorocyclohexane Lipid Adjusted; Retinyl palmitate (ug/dL); Trans 9-octadecenoic acid (uM); 1,2,3,7,8,9-Hexachlorodibenzofuran (hxcdf) (fg/g); 1,2,3,4,7,8,9-Heptachlorodibenzofuran (Hpcdf) (fg/g); PCB87 (ng/g); and Red cell count SI. In some embodiments, the two or more biomarkers are selected from the group: Glucose, serum (mg/dl); Creatinine (mg/dl); Lactate dehydrogenase LDH (U/L); Uric acid (mg/dl); Blood lead (ug/dl); Homocysteine(umol/L); Vitamin A (ug/dl); Fasting Glucose (mg/dl); GGT: SI (U/L); Total cholesterol (mg/dl); Vitamin E (ug/dl); Chloride: SI (mmol/L); AST: SI (U/L); Sodium: SI (mmol/L); PCB180 (ng/g); Cholesterol (mg/dl); PCB170 (ng/g); Alkaline phosphatase (U/L) and glycohemoglobin, glucose serum, glycohemoglobin, creatine, lactate dehydrogenase, uric acid, blood lead, homocysteine, vitamin A, fasting glucose, gamma glutamyltransferase (GGT), total cholesterol, Vitamin E, chloride, aspartate aminotransferase (AST), sodium, and 2,2′,3,4,4′,5,5′-heptachlorobiphenyl (PCB180), glucose serum, glycohemoglobin, creatine, lactate dehydrogenase, uric acid, blood lead, homocysteine, vitamin A, fasting glucose, gamma glutamyltransferase (GGT), and total cholesterol. In some embodiments, biomarkers characteristic of aging are selected from: glucose serum, glycohemoglobin, creatine, lactate dehydrogenase, uric acid, melatonin and blood lead.

19. The method of claim 1, wherein such method is implemented in a computer.

20. A tangible medium, configured with instructions that when executed cause a processor to perform the method of claim 1.
